SEQUENCE LISTING (5 sequences)

SEQ ID NO: 1 - DNA, 21 bp, Artificial Sequence
  Chemically synthesized
ctcttcagcattattccagtg

SEQ ID NO: 2 - DNA, 20 bp, Artificial Sequence
  Chemically synthesized
cgagttccttgatgtagttc

SEQ ID NO: 3 - DNA, 19 bp, Artificial Sequence
  Chemically synthesized
cggcaggcaagatagacag

SEQ ID NO: 4 - DNA, 20 bp, Artificial Sequence
  Chemically synthesized
tgggctccaggagaatcttt

SEQ ID NO: 5 - DNA, 17 bp, Artificial Sequence
  chemically synthesized
caggcaagatagacagc

(19) United States
(12) Patent Application Publication
(10) Pub. No.: US 2012/0295932 A1
(43) Pub. Date: Nov. 22, 2012

(54) METHOD FOR THE TREATMENT OF CANCER

(75) Inventors: Cristiano Ferlini, Rome (IT); Marisa Mariani, Sulmona (IT); Shohreh Shahabi, New York, NY (US)

(73) Assignee: Western Connecticut Health Network, Inc., Danbury, CT (US)

(21) Appl. No.: 13/329,707

(22) Filed: Dec. 19, 2011

Related U.S. Application Data
(63) Continuation-in-part of application No. 13/109,128, filed on May 17, 2011.

(51) Int. Cl. A61K 31/437; A61P 35/00; A61K 31/282; C12Q 1/68; G01N 33/574

(52) U.S. Cl. 514/283; 435/6.12; 435/7.23; 514/492

(57) ABSTRACT

Provided is a method for determining the prognosis of cancer in a female patient with a solid tumor. Also provided is a method of sensitizing a cancer cell from a solid tumor from a male patient other than a prostate cancer cell to treatment with a chemotherapeutic agent comprising administering an effective amount of
(a)
   (i) at least one antiandrogen;
   (ii) at least one inhibitor of androgen synthesis; or
   (iii) a combination of at least one antiandrogen and at least one inhibitor of androgen synthesis; and
(b) at least one chemotherapeutic agent other than an antiandrogen or an inhibitor of androgen synthesis for the treatment of cancer characterized by a solid tumor, other than prostate cancer, in a male patient. The antiandrogen, the inhibitor of androgen synthesis, or the combination of both may be administered before, after, or at the same time as the chemotherapeutic agent.

### CROSS-REFERENCE TO RELATED APPLICATIONS

This application is a continuation-in-part of application Ser. No. 13/109,128, filed May 17, 2011, which is hereby incorporated by reference in its entirety.

### FIELD OF THE INVENTION

The invention relates to a method for the prognosis of cancer in females and a method for the treatment of cancer in males.

### BACKGROUND OF THE INVENTION

Colorectal cancer is one of the deadliest diseases in western countries. Its incidence in the United States is about 140,000 novel cases per year. It is the third leading cause of cancer-related deaths when men and women are considered separately, and the second when both genders are combined. It is expected to cause about 51,370 deaths (26,580 in men and 24,790 in women) during 2011. Although this number has improved in the last two decades, particularly due to a wider use of early surgery and endoscopic techniques combined with screening campaigns, there is an urgent need to improve our knowledge regarding this disease, particularly for those patients in which diagnosis is performed at an advanced stage. In this case, surgery alone is not sufficient and chemotherapy is needed, but its success will be largely dependent on biologic factors whose nature is still elusive. In fact, the main obstacle to a successful treatment in this disease, like in the other solid tumors, is represented by drug-resistance. By looking at the mechanisms underlying this phenomenon, several reports have indicated the strict relationship with genetic stability of the tumor and the selective pressure of tumor microenvironment. This explains why many biomarkers capable of predicting the response to chemotherapy are linked to pathways of cell survival which are activated as adaptive responses to stressing microenvironments. These cell survival pathways make it possible for cancer cells to survive also in conditions featured by poor nutrient supply and low oxygen levels. Unfortunately, the same pathways as collateral damage can be responsible for the resistance to radio- and chemotherapy, thus explaining failure of these patients upon conventional treatments in colorectal cancer as well as in other tumors. The failure to predict which patients will respond to a given treatment will expose them to severe, adverse side effects linked to chemotherapy also in the case when, being non respondent to chemotherapeutics, the patient will not benefit.

Among these survival pathways, a prominent role is played by the overexpression of class III β-tubulin (TUBB3). Although originally discovered as a mechanism of drug resistance to taxanes (Derry W B, Wilson L, Khan I A, Luduena R F, Jordan M A. Taxol differentially modulates the dynamics of microtubules assembled from unfractionated and purified beta-tubulin isotypes. Biochemistry 1997, 36:3554-3562), recent studies have demonstrated that TUBB3 is involved in an adaptive response to low oxygen levels and poor nutrient supply in a growing number of solid tumors. This explains TUBB3 involvement in drug resistance independently of whether the disease is treated with a regimen including a microtubule targeting agent or not. By looking at the protein sequence, one way to explain TUBB3 function is that this protein is able to form cysteine disulphide bridges with other proteins involved in the adaptive response to oxidative stress, thereby allowing their incorporation into microtubules. As compared to the constitutive isotypes, a peculiar feature of TUBB3 are the switches Cysteine/Serine and Alanine/Cysteine at position 124 and 239, respectively. The regions around positions 124 and 239 are resistant and sensitive to oxidation, respectively. Therefore, Cysteine 124 can form stable complexes in oxidative stress conditions, while this does not occur at position 239. Remarkably, such a feature is shared also with class V β-tubulin isotype (TUBB6), a protein for which no clinical studies are reported in colorectal cancer. Translational studies on TUBB6 are lacking for the absence of a specific commercial antibody available for analysis in paraffin-embedded specimens.

Previous studies are divided about the relationship between outcome and gender in advanced colorectal cancer, whose treatment involves surgery and adjuvant chemotherapy. Aside from two studies reporting that a greater proportion of women had curative surgery for colorectal cancer compared with men (McArdle C S, McMillan D C, Hole D J. Male gender adversely affects survival following surgery for colorectal cancer. Br J Surg. 2003, 90:711-5; Roetzheim R G, Pal N, Gonzalez E C, Ferrante J M, Van Durme D J, Krischer J P. Effects of health insurance and race on colorectal cancer treatments and outcomes. Am J Public Health 2000, 90:1746-54), the majority of investigations reported comparable curative surgery rates (Chapuis P H, Dent O F, Fisher R, Newland R C, Pheils M T, Smyth E, Colquhoun K. A multivariate analysis of clinical and pathological variables in prognosis after resection of large bowel cancer. Br J Surg 1985, 72:698-702; Kune G A, Kune S, Field B, White R, Brough W, Schellenberger R, Watson L F. Survival in patients with large-bowel cancer. A population-based investigation from the Melbourne Colorectal Cancer Study. Dis Colon Rectum 1990, 33:938-46; Wichmann M W, Müler C, Hornung H M, Lau-Werner U, Schildberg F W; Colorectal Cancer Study Group. Gender differences in long-term survival of patients with colorectal cancer. Br J Surg 2001, 88:1092-8; Koo J H, Jalaludin B, Wong S K, Kneebone A, Connor S J, Leong R W. Improved survival in young women with colorectal cancer. Am J Gastroenterol 2008, 103:1488-95; Newland R C, Dent O F, Lyttle M N, Chapuis P H, Bokey E L. Pathologic determinants of survival associated with colorectal cancer with lymph node metastases. A multivariate analysis of 579 patients. Cancer 1994, 73:2076-82). Only one report revealed women were less likely to undergo abdominoperineal resection due to differences in pelvic anatomy (Beart R W, Steele G D Jr, Menck H R, Chmiel J S, Ocwieja K E, Winchester D P. Management and survival of patients with adenocarcinoma of the colon and rectum: a national survey of the Commission on Cancer. J Am Coll Surg 1995, 181:225-36). Also the impact of gender on efficacy of adjuvant chemotherapy is controversial. A meta-analysis of six chemotherapy studies with 786 patients demonstrated no sex difference in survival despite women requiring more dose reductions due to increased side-effects (Sloan J A, Goldberg R M, Sargent D J, Vargas-Chanes D, Nair S, Cha S S, Novotny P J, Poon M A, O'Connell M J, Loprinzi C L. Women experience greater toxicity with fluorouracil-based chemotherapy for colorectal cancer. J Clin Oncol 2002, 20:1491-8). However, two studies reported sex differences in survival following chemotherapy (Elsaleh H, Joseph D, Grieu F, Zeps N, Spry N, Iacopetta B. Association of tumour site and sex with survival benefit from adjuvant chemotherapy in colorectal cancer. Lancet 2000, 355:1745-50; Watanabe T, Wu T T, Catalano P J, Ueki T, Satriano R, Haller D G, Benson A B 3rd, Hamilton S R. Molecular predictors of survival after adjuvant chemotherapy for colon cancer. N Engl J Med 2001, 344:1196-206).

Despite the scattered evidence of a gender-related differential prognosis in colorectal cancer, the role of androgens in this process has been marginal, if not completely neglected, since the vast majority of the reports focus on the activity of estrogens as responsible for such protective effect. This concept stems from the clinical evidence that hormone replacement therapy in postmenopausal women induce a 44% reduction in colorectal cancer incidence as compared to the placebo control (Ritenbaugh C, Stanford J L, Wu L, Shikany J M, Schoen R E, Stefanick M L, Taylor V, Garland C, Frank G, Lane D, Mason E, McNeeley S G, Ascensao J, Chlebowski R T; Women's Health Initiative Investigators. Conjugated equine estrogens and colorectal cancer incidence and survival: the Women's Health Initiative randomized clinical trial. Cancer Epidemiol Biomarkers Prey 2008, 17:2609-18).

### SUMMARY OF THE INVENTION

Provided is a method for determining the prognosis of a cancer characterized by a solid tumor in a female patient comprising determining the percentage of solid tumor cells expressing TUBB3 protein and the percentage of solid tumor cells expressing TUBB6 protein. Detection of TUBB6 protein expression in a percentage of solid tumor cells that is less than a median percentage expression for TUBB6 protein and detection of TUBB3 protein expression in a percentage of solid tumor cells that is less than the median percentage expression for TUBB3 protein indicates that the patient has a good prognosis; and detection of TUBB6 protein expression in a percentage of solid tumor cells of the female patient that is equal to or greater than the median percentage expression for TUBB6 protein and/or detection of TUBB3 protein expression in a percentage of solid tumor cells of the female patient that is equal to or greater than the median percentage expression for TUBB3 protein indicates that the patient has a poor prognosis. In some embodiments the median percentage expression of TUBB3 protein is determined by determining the percentage of solid tumor cells in a solid tumor sample expressing TUBB3 protein in a plurality of solid tumors, of essentially the same tumor type as the solid tumor of claim 1, taken from patients of both genders who are suffering from the cancer and determining the median; and the median percentage expression for TUBB6 protein is determined by determining the percentage of solid tumor cells in a solid tumor sample expressing TUBB6 protein in a plurality of solid tumors, of essentially the same tumor type as the solid tumor of claim 1, taken from patients of both genders who are suffering from the cancer and determining the median. In some embodiments, the cancer is colorectal cancer or lung cancer. In some embodiments the cancer is colorectal cancer, the median percentage expression for TUBB3 protein is 35% and the median percentage expression for TUBB6 protein is 60%. In further embodiments, the solid tumor sample from the patient is a paraffin-embedded sample. In yet further embodiments, the female patient is human. In yet further embodiments, the cancer has a gender-specific prognosis.

Provided is a method for treating a female patient with a solid tumor comprising administering a chemotherapeutic agent to a female patient following prognosis according to the method of the first embodiment.

Provided is a method of sensitizing a cancer cell of a solid tumor of a male patient, other than a prostate cancer cell, to treatment with a chemotherapeutic agent comprising administering to the patient an effective amount of

(a)

(i) at least one antiandrogen;

(ii) at least one inhibitor of androgen synthesis; or

(iii) a combination of at least one antiandrogen and at least one inhibitor of androgen synthesis; and

(b) at least one chemotherapeutic agent other than an antiandrogen or an inhibitor of androgen synthesis. In some embodiments the cancer is colorectal cancer or lung cancer. In some embodiments the chemotherapeutic agent is oxaliplatin or irinotecan. In further embodiments the patient is human. In yet further embodiments the cancer cell constitutively expresses TUBB3 protein and TUBB6 protein, wherein the survival rate of the cancer is worse for males than for females. In further embodiments, the chemotherapeutic agent is administered to the cancer cell before the antiandrogen and/or the inhibitor of androgen synthesis. In further embodiments, the antiandrogen and/or the inhibitor of androgen synthesis is administered to the cancer cell before the chemotherapeutic agent. In yet further embodiments the chemotherapeutic agent and the antiandrogen and/or the inhibitor of androgen synthesis are administered to the cancer cell at the same time. In preferred embodiments the antiandrogen is bicalutamide, flutamide, nicalutamide, spironolactone, cyproterone acetate or finasteride. In further preferred embodiments the inhibitor of androgen synthesis is an inhibitor of CYP17 or AKR1C3. In yet further preferred embodiments the inhibitor of CYP17 is ketoconazole, abiraterone, abiraterone acetate, VN/124(TOK-001) or TAK-700, or salts thereof. In preferred embodiments the inhibitor of AKR1C3 is an indomethacin analog. In further preferred embodiments the indomethacin analog is N-(4-chlorobenzoyl)-melatonin.

The invention is also directed to the use in medicine of an effective amount of

(a)

(i) at least one antiandrogen;

(ii) at least one inhibitor of androgen synthesis; or

(iii) a combination of at least one antiandrogen and at least one inhibitor of androgen synthesis; and

(b) at least one chemotherapeutic agent other than an antiandrogen or an inhibitor of androgen synthesis for the treatment of cancer characterized by a solid tumor, other than prostate cancer, in a male patient.

The invention is also directed to the use of

(a)

(i) at least one antiandrogen;

(ii) at least one inhibitor of androgen synthesis; or
(iii) a combination of at least one antiandrogen and at least one inhibitor of androgen synthesis; and

(b) at least one chemotherapeutic agent other than an antiandrogen or an inhibitor of androgen synthesis, in the preparation of a medicament for treatment of cancer characterized by a solid tumor, in a male patient.

The invention is also directed to the use of

(a)

(i) at least one antiandrogen;

(ii) at least one inhibitor of androgen synthesis; or

(iii) a combination of at least one antiandrogen and at least one inhibitor of androgen synthesis; and

(b) at least one chemotherapeutic agent other than an antiandrogen or an inhibitor of androgen synthesis, for use in in the preparation of a medicament for treatment of cancer characterized by a solid tumor, in a male patient.

Also provided is a kit for determining the prognosis of a cancer in a female patient with a solid tumor comprising a set of reagents that specifically detects the protein expression levels of TUBB3 protein and TUBB6 protein in cells from the solid tumor, and instructions for using the kit for evaluating the prognosis of cancer characterized by a solid tumor in a female patient.

As envisioned in the present invention with respect to the disclosed compositions of matter and methods, in one aspect the embodiments of the invention comprise the components and/or steps disclosed therein. In another aspect, the embodiments of the invention consist essentially of the components and/or steps disclosed therein. In yet another aspect, the embodiments of the invention consist of the components and/or steps disclosed therein.

### BRIEF DESCRIPTION OF THE DRAWINGS

FIG. 1 illustrates a Kaplan-Meier analysis of 147 colorectal cancer patients recruited in a clinical study, grouped by clinical stage according to the American Joint Committee on Cancer (AJCC) criteria. For these 147 patients, the full clinical history was available and the median follow up was three years. See Example 1. Stage 1 and Stage 2 patients exhibited a similar outcome, with risk of death after 5 years of about 5%. The risk is increased to 30% for stage 3 patients. The risk of death is 100% for patients at stage 4.

FIG. 2 illustrates a bar chart reporting the percentage of expression of TUBB3 and TUBB6 in 180 colorectal cancer patients recruited in a study. See Example 1. Patients were stratified in 4 groups according to the percentage of staining of TUBB3 (cutoff 35%) or TUBB6 (cutoff 60%). TUBB3 percentage staining equal to or greater than 35% is considered “positive” and TUBB6 percentage staining equal to or greater than 60% is considered “positive.” Values for TUBB3 and TUBB6 below those cutoff points are considered “negative.” Groups 1, 2, 3 and 4 were double negative, single TUBB3+, single TUBB6+ and double positive, respectively, for TUBB3 and TUBB6 protein expression. There was a tendency to co-express the two antigens with 60 and 59 patients belonging to group 1 and 4 respectively, therefore making statistically significant the probability of co-expression (Pearson's χ²=18.7, p<0.001).

FIG. 3 illustrates a Kaplan-Meier analysis of the 147 colorectal cancer patients recruited in the clinical study of Example 1 grouped according to TUBB3/TUBB6 staining There is no statistically significant difference in the outcome in any of the groups.

FIG. 4 illustrates a Kaplan-Meier analysis of the 147 colorectal cancer patients recruited in the clinical study of Example 1 grouped by gender. As compared to the females in the study, males exhibited a poorer outcome and a faster progression to death. The difference was statistically significant (p=0.0188).

FIG. 5 illustrates in A: Kaplan-Meier analysis of the 82 male colorectal cancer patients recruited in the clinical study of Example 1, grouped according to TUBB3/TUBB6 staining. No difference in the outcome is evident; FIG. 5B: Kaplan-Meier analysis of the 65 female colorectal cancer patients recruited in the clinical study grouped according to TUBB3/TUBB6 staining Double negative patients all survived (18/18) and exhibited a different outcome as compared to the other three groups with statistical significance (p value=0.022).

FIG. 6 illustrates in A-D: a dot chart showing the percentage of expression of TUBB6 (FIG. 6A and FIG. 6C) and TUBB3 (FIG. 6B and FIG. 6D) in the 65 female colorectal patients (FIG. 6A and FIG. 6B) and in the 82 male colorectal patients (FIG. 6C and FIG. 6D). Values are shown in two different columns reporting data for patients without (left column) or with (right column) metastatic disease. The diamond in each plot represents the 95% confidence interval and the line in the middle of the diamond represents the mean value for each column.

FIG. 7 illustrates a bar chart showing the gene expression value of TUBB6 quantified using qPCR analysis in a panel of 22 colorectal cancer cell lines. See Example 2. Cell lines originating from males have higher expression values of TUBB6 as compared to the cell lines from females. This phenomenon is not present for TUBB3.

FIG. 8 illustrates in A-D: Line chart representing qPCR analysis of TUBB3 (FIG. 8A and FIG. 8C) and TUBB6 (FIG. 8B and FIG. 8D) in male origin cell lines (FIG. 8A and FIG. 8B) and in female origin cell lines (C and D). The same panel of 22 colorectal cancer cell lines of FIG. 7 was employed. See Example 2. Left column displays values for control cells while right column represent the values after 72 hours of serum starvation. FIGS. 8E-F: The values reported in A-D were used to generate the dot chart shown in E and F where the results for TUBB3 and TUBB6 are depicted, respectively. Each cell line was normalized for the control and the percent increase in gene expression is shown. The horizontal line indicates the mean value for males and females.

FIG. 9 illustrates in A-B: Dot chart representing the expression value for TUBB3 (FIG. 9A) and TUBB6 (FIG. 9B) at the protein level after serum starvation. The same panel of 22 colorectal cancer cell lines of FIG. 7 was employed. See Example 2. Densitometric analysis was performed in three independent Western blots of control and serum-starved cells. Data coming from densitometric analysis was normalized for the loading control and the increase with respect to control was averaged and is represented in the chart. In the left column and right column data points are for cell lines originating from males and females, respectively. The horizontal line indicates the mean for each group. FIG. 9C: A representative Western blot for 4 cell lines, two from males (SW837 and SW620) and two from females for TUBB3 and TUBB6. RNA-binding protein HuR was used as loading control.

FIG. 10 illustrates a dot chart representing the percent of live cells after 72 h of serum starvation. The same panel of 22 colorectal cancer cell lines of FIG. 7 was employed. Each data point is the average of three independent experiments. The number of live cells was counted in the control and in serum starved cells and normalized as percent of control. In the left and right columns data for male and female cell lines are shown, respectively. The line indicates the mean for each group. Male cell lines have better survival ability after 72 h of serum starvation.

FIG. 11 is a bar chart showing the gene expression value of androgen receptor (AR, isoforms 1 and 2) quantified using qPCR analysis in the panel of 22 colorectal cancer cell lines. The same cell lines in both genders exhibiting the highest value of TUBB6, highly express AR (Sw480, Caco-2 and Colo-320).

FIG. 12 illustrates a representative clonogenic assay performed in SW480 cells. Cells were treated with the vehicle (DMSO 0.2%, Control), with bicalutamide (CDX) 20 μM, with oxaliplatin (concentration points 750, 1500, 3000 and 6000 nM) and with the combination of the two. Drug treatment lasted 24 hours. Thereafter cells were counted and plated at a density of 350 cells/dish. The colonies were stained with methylene blue and analyzed as shown in FIG. 12.

FIG. 13 illustrates in A-D: a line chart reporting the effect of the combination oxaliplatin/bicalutamide (FIGS. 13A and 13C) and SN38/bicalutamide (FIGS. 13B and 13D) in SW480 male (FIGS. 13A and 13B) and RKO female (FIGS. 13C and 13D) colorectal cancer cells. Cells were treated for 12 hours with either oxaliplatin or SN38 at the indicated concentrations and then washed and kept for an additional 72 hours in the presence of bicalutamide (20 μM). At the end of the treatment cells were washed, trypsinized and plated in Petri dish at 350 cells per dish. After 14 days the cell colonies were stained with methylene blue and counted with automatic imaging software (Carestream, Image Station 4000R PRO). Each data point refers to the mean of a triplicate experiment while error bars are the corresponding standard deviations.

FIG. 14 illustrates in A-B: chemical structures of ketoconazole and abiraterone, which are CYP17 inhibitors that are used in the clinic (FIG. 14A) and of VN/124-1 (TOK-0001) and TAK-700, which are CYP17 inhibitors that are in clinical trials (FIG. 14B).

FIG. 15 illustrates in A-B: Kaplan-Meier analysis showing that male patients carrying the G allele of the RS743572 single nucleotide polymorphism (CYP17) exhibited the highest risk of death (odds ratio (OR) 10.7, 95% confidence intervals (CI), 1.35-86.1), while in females this deleterious effect of the G allele was completely absent (OR 0.98, 95% CI, 0.33-2.87).

### DEFINITIONS

As used herein, each of the following terms has the meaning associated with it in this section.

The articles “a” and “an” are used herein to refer to one or to more than one (i.e. to at least one) of the grammatical object of the article. By way of example, “an element” means one element or more than one elements.

The term “about” will be understood by persons of ordinary skill in the art and will vary to some extent depending on the context in which it is used. As used herein, “about” is meant to encompass variations of ±20% or ±10%, more preferably ±5%, even more preferably ±1%, and still more preferably ±0.1%.

The term “in vitro method,” as used herein, refers to a method carried out outside of a living organism as opposed to an “in vivo method” which is a method carried out inside or on a living organism.

The term “antiandrogen,” as used herein, refers to a treatment aimed at inhibiting the biological effect mediated by androgen receptor. This includes but is not limited to chemical agents such as bicalutamide, flutamide, nicalutamide, spironolactone, cyproterone acetate, dutasteride, bexlosteride, izonsteride, turosteride, epristeride and finasteride. Antiandrogen may also include thereapeutic strategies aimed at inhibiting the expression of androgen receptor such as SiRNA targeting androgen receptor.

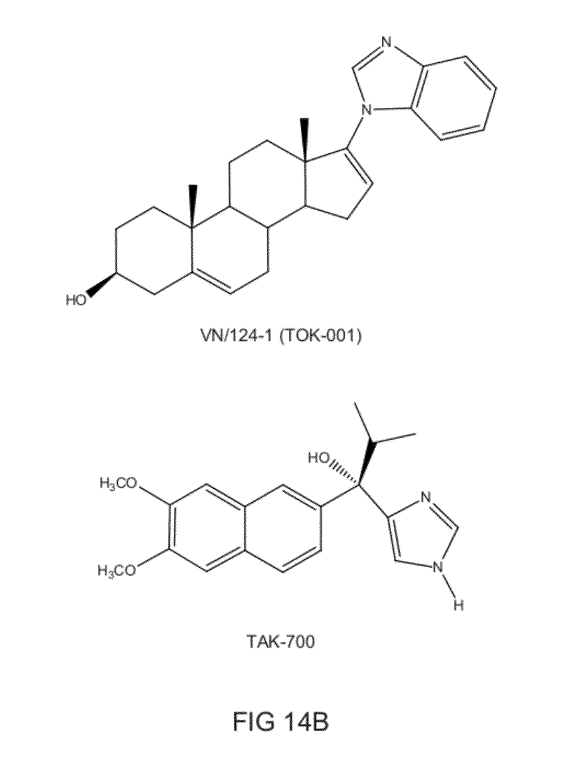

The term “inhibitor of androgen synthesis,” as used herein, refers to a chemical or biological agent that inhibits the synthesis of an androgen. In some embodiments, the inhibitor of androgen synthesis is an inhibitor of an enzyme involved in androgen synthesis. In some embodiments, the inhibitor of androgen synthesis is an inhibitor of 17α-hydroxy/17,20-lyase (CYP17). Examples of inhibitors of CYP17 include but are not limited to abiraterone, abiraterone acetate, ketoconazole, VN/124-1(3β-hydroxy-17-(1H-benzimidazole-1-yl)androsta-5,16-diene, now called TOK-001) or TAK-700 [(1S)-1-(6,7-dimethoxy-2-naphthyl)-1-(1H-imidazol-4-yl)-2-methylpropan-1-ol], whose structures are illustrated in FIGS. 14A-B, or salts thereof (Vasaitis T S, Bruno R D and Njar VCO, CYP17 inhibitors for prostate cancer therapy, J. Steroid Biochem. Mol. Biol., 2011, 125:23-31, which is hereby incorporated by reference in its entirety). In some embodiments the inhibitor of androgen synthesis is an inhibitor of AKR1C3. In further embodiments, the inhibitor of AKR1C3 is an indomethacin analog. In yet further embodiments, the indomethacin analog is N-(4-chlorobenzoyl)-melatonin. (Byrns M C, Steckelbroeck S and Penning T M, An indomethacin analogue, N-(4-chlorobenzoyl)-melatonin, is a selective inhibitor of aldo-keto reductase 1C3 (type 2 3 α-HSD, type 5 17β-HSD, and prostaglandin F synthase), a potential target for the treatment of hormone dependent and hormone independent malignancies, Biochem. Pharmacol., 2007, 75(2):484-493).

The term “chemotherapeutic agent,” as used herein, refers to a chemical agent used for treating various forms of cancer generally by directly killing the cancer cells. A chemotherapeutic agent may also be known as an “antineoplastic agent.” Examples of chemotherapeutic agents include but are not limited to oxaliplatin and irinotecan. Additional examples include paclitaxel, topotecan, doxorubicin and cisplatin.

The term “prognosis,” as used herein, refers to a prediction of the probable outcome and course of a disease or condition.

The term “outcome,” as used herein, refers to the course of a disease or condition; or the rate of survival of a patient suffering from the disease or condition.

The term “good prognosis,” as used herein, refers to greater than average likelihood of survival for a patient suffering from the disease or condition as compared to other members of the same gender suffering from the same disease or condition.

The term “poor prognosis,” as used herein, refers to a less than average likelihood of survival for a patient suffering from the disease or condition as compared to other members of the same gender suffering from the same disease or condition.

The term “sensitizing,” as used herein, refers to making the cell(s) more sensitive to the effects of another drug.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies that may be used in the practice of the present invention may exist in a variety of forms including, for example, polyclonal antibodies, monoclonal antibodies, Fv, Fab and F(ab)₂, as well as single chain antibodies and humanized antibodies (Harlow et al., 1999, Using Antibodies: A Laboratory Manual, Cold Spring Harbor Laboratory Press, New York; Harlow et al., 1989, Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

The term “control” or “reference standard” describes a material comprising a level of TUBB3 or TUBB6 polypeptide, such that the control or reference standard may serve as a comparator against which a sample can be compared.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence which is identical to the mRNA sequence and is usually provided in sequence listings, and the non-coding strand, used as the template for transcription of a gene or cDNA, can be referred to as encoding the protein or other product of that gene or cDNA.

“Gene expression” or “expression” as used herein refers to the process by which information from a gene is made into a functional gene product, such as RNA or protein. Thus, the “level of expression” of a gene product of a marker gene, in a sample of interest, refers to the level of RNA, particularly the level of mRNA, or the level of the encoded protein, and is not intended to be limited to either, unless so specified. “Protein expression,” as used herein refers to the level of protein.

The term “gene” refers to a nucleic acid (e.g., DNA) sequence that comprises coding sequences necessary for the production of a polypeptide, precursor, or RNA (e.g., mRNA, rRNA, tRNA). The term “gene” encompasses both cDNA and genomic forms of a gene.

As used herein, an “instructional material” includes a publication, a recording, a diagram, or any other medium of expression, which can be used to communicate the usefulness of the invention in the kit for determining the progression of a disease. The instructional material of the kit of the invention may, for example, be affixed to a container, which contains a reagent of the invention or be shipped together with a container, which contains a reagent. Alternatively, the instructional material may be shipped separately from the container with the intention that the instructional material and the reagent be used cooperatively by the recipient.

“Measuring” or “measurement,” or alternatively “detecting” or “detection,” or alternatively “determining” or “determine” means assessing the presence, absence, quantity or amount of either a given substance within a sample, including the derivation of qualitative or quantitative concentration levels of such substances.

“Sample” or “biological sample” as used herein means a biological material that contains a substance under assay for determination of gene product expression level. The sample may contain any biological material suitable for detecting TUBB3 or TUBB6, and may comprise cellular and/or non-cellular material.

The term “solid support,” “support,” and “substrate” as used herein are used interchangeably and refer to a material or group of materials having a rigid or semi-rigid surface or surfaces. In one embodiment, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations. See U.S. Pat. No. 5,744,305 for exemplary substrates.

“Specifically binds” as used herein in the context of an antibody or an aptamer refers to antibody or aptamer binding to a predetermined antigen with a preference that enables the antibody to be used to distinguish the antigen from others to an extent that permits the detection of the target antigens described herein.

### DETAILED DESCRIPTION OF THE INVENTION

Provided is a method for determining the prognosis of cancer in a female patient with a solid tumor comprising determining the percentage of solid tumor cells expressing TUBB3 protein and the percentage of solid tumor cells expressing TUBB6 protein in a solid tumor sample from the patient. Detection of TUBB6 protein expression in a percentage of solid tumor cells of the female patient that is less than the median percentage expression for TUBB6 protein and detection of TUBB3 protein expression in a percentage of solid tumor cells of the female patient that is less than the median percentage expression for TUBB3 protein indicates a patient with good prognosis; and detection of TUBB6 protein expression in a percentage of solid tumor cells of the female patient that is equal to or greater than the median percentage expression for TUBB6 protein and/or detection of TUBB3 protein expression in a percentage of solid tumor cells of the female patient that is equal to or greater than the median percentage expression for TUBB3 protein, indicates a patient with poor prognosis. In some embodiments the median percentage expression of TUBB3 protein is determined by determining the percentage expression for TUBB3 protein in a plurality of solid tumors, of essentially the same tumor type as the solid tumor of claim 1, taken from patients of both genders who are suffering from the cancer and determining the median; and the median percentage expression for TUBB6 protein is determined by determining the percentage expression for TUBB6 protein in a plurality of solid tumors, of essentially the same tumor type as the solid tumor of claim 1, taken from patients of both genders who are suffering from the cancer and determining the median. In some embodiments, the cancer is colorectal cancer or lung cancer. In some embodiments the cancer is colorectal cancer, the median percentage expression for TUBB3 protein is 35% and the median percentage expression for TUBB6 protein is 60%. In further embodiments, the solid tumor sample from the patient is a paraffin-embedded sample. In yet further embodiments, the female patient is human. In yet further embodiments, the cancer has a gender-specific prognosis.

Provided is a method for treating a female patient with a solid tumor comprising administering a chemotherapeutic agent to a female patient following prognosis according to the method of the first embodiment.

Provided is a method of sensitizing a cancer cell of a solid tumor of a male patient, other than a prostate cancer cell, to treatment with a chemotherapeutic agent comprising administering to the patient an effective amount of

(a)

(i) at least one antiandrogen;

(ii) at least one inhibitor of androgen synthesis; or

(iii) a combination of at least one antiandrogen and at least one inhibitor of androgen synthesis; and

(b) at least one chemotherapeutic agent other than an antiandrogen or an inhibitor of androgen synthesis. In some embodiments the cancer is colorectal cancer or lung cancer. In some embodiments the chemotherapy is oxaliplatin or irinotecan. In further embodiments the patient is human. In yet further embodiments the cancer cell from a solid tumor from a male patient consitutively expresses TUBB3 protein and TUBB6 protein, wherein the survival rate of the cancer is worse for males than for females. In further embodiments the chemotherapy is administered to the cancer cell before the antiandrogen and/or the inhibitor of androgen synthesis. In further embodiments the antiandrogen and/or the inhibitor of androgen synthesis is administered to the cancer cell before the chemotherapy. In yet further embodiments the chemotherapy and the antiandrogen and/or the inhibitor of androgen synthesis are administered to the cancer cell at the same time. In preferred embodiments the antiandrogen is bicalutamide, flutamide, nicalutamide, spironolactone, cyproterone acetate or finasteride. In further preferred embodiments the inhibitor of androgen synthesis is an inhibitor of CYP17. In yet further preferred embodiments the inhibitor of CYP17 is ketoconazole, abiraterone, abiraterone acetate, VN/124(TOK-001) or TAK-700, or salts thereof.

The expression of TUBB6 is higher in males than it is in females. See Example 2. We have observed a correlation between the expression of androgen receptor and the expression of TUBB6 in males. See Example 3. We have also shown that treatment with an antiandrogen can sensitize a cell of a solid tumor in a male patient to chemotherapy. See Example 3. Without wishing to be bound by any theory, treatment with an antiandrogen counteracts the deleterious effect of high androgens in males patients with a solid tumor. See Example 3.

We have also observed a correlation between the presence of high levels of androgens and an increased risk of death from colorectal cancer in males. See Example 4. This correlation was absent in females. Treatment with an inhibitor of androgen synthesis will lead to a decreased risk of death from colorectal cancer in males. Treatment of a cell of a solid tumor in a male patient with any agent that leads to a decrease in androgen levels can sensitize the cell to chemotherapy. Treatment with an agent that leads to a decrease in androgen levels will lead to a decreased risk of death and an improved outcome. Examples of agents that lead to a decrease in androgen levels include, but are not limited to antiandrogens or inhibitors of androgen synthesis.

An enzyme that is involved in androgen synthesis that can be targeted by inhibitors of androgen synthesis is CYP17. CYP17 is an enzyme which is capable of directing steroid synthesis towards the synthesis of androgens, and has two key enzymatic activities:

(i) the 17α-hydroxylase activity typically converts pregnenolone to 17α-hydroxypregnenolone and converts progesterone to 17α-hydroxyprogesterone; and

(ii) the C17,20-lyase activity converts 17α-hydroxypregnenolone to DHEA and 17α-hydroxyprogesterone to androstenedione.

Several single nucleotide polymorphisms (SNPs) of CYP17 have been characterized. The RS743572 SNP most represented in the population carries an A allele in the 5′-UTR of the CYP17A1 gene. The G allele has an additional binding site for the Sp-1 transcription factor, which leads to increased transcription of the gene and enhanced levels of androgens (Habuchi T, Liqing Z, Suzuki T, Sasaki R, Tsuchiya N, Tachiki H, Shimoda N, Satoh S, Sato K, Kakehi Y, Kamoto T, Ogawa O, and Kato T, Increased risk of prostate cancer and benign prostatic hyperplasia associated with a CYP17 gene polymorphism with a gene dosage effect, Cancer Res., 2000, 60:5710-5713; Lunn R M, Bell D A, Mohler L and Taylor J A, Prostate cancer risk and polymorphism in 17 hydroxylase (CYP 17) and steroid reductase (SRD5A2), Carcinogenesis, 1999, 20(9):1727-1731). The “A allele” that we are referring to corresponds to the “A1” allele in Habuchi et al. and our “G allele” corresponds to the “A2” allele of Habuchi et al. Men carrying the A2 allele have been shown to have increased levels of serum bioavailable androgens (Zmuda J M, Cauley J A, Kuller L H and Ferrell R E, J. Bone and Mineral Research, 2001, 16(5):911-917).

Another enzyme that is involved in androgen synthesis that can be targeted by inhibitors of androgen synthesis is AKR1C3. In preferred embodiments, the inhibitor of AKR1C3 is an indomethacin analog. In yet further embodiments, the indomethacin analog is N-(4-chlorobenzoyl)-melatonin. (Byrns M C, Steckelbroeck S and Penning T M, An indomethacin analogue, N-(4-chlorobenzoyl)-melatonin, is a selective inhibitor of aldo-keto reductase 1C3 (type 2 3 α-HSD, type 5 17β-HSD, and prostaglandin F synthase), a potential target for the treatment of hormone dependent and hormone independent malignancies, Biochem. Pharmacol., 2007, 75(2):484-493).

The cancers envisioned for prognosis or treatment by the inventions disclosed herein comprise a broad range of cancer types including but not limited to the following: ovarian cancer, cervical cancer, breast cancer, testicular cancer, lung cancer, renal cancer, colorectal cancer, skin cancer and brain cancer.

More particularly, cancers envisioned for prognosis or treatment in either female or male patients by the inventions disclosed herein include, but are not limited to, the following:

cardiac cancers, including, for example sarcoma, e.g., angiosarcoma, fibrosarcoma, rhabdomyosarcoma, and liposarcoma; myxoma; rhabdomyoma; fibroma; lipoma and teratoma;

lung cancers, including, for example, brochogenic carcinoma, e.g., squamous cell, undifferentiated small cell, undifferentiated large cell, and adenocarcinoma; alveolar and bronchiolar carcinoma; bronchial adenoma; sarcoma; chondromatous hartoma; and mesothelioma;

gastrointestinal cancer, including, for example, cancers of the esophagus, e.g., squamous cell carcinoma, adenocarcinoma, and leiomyosarcoma; cancers of the stomach, e.g., carcinoma, and leiomyosarcoma; cancers of the pancreas, e.g., ductal adenocarcinoma, insulinoma, glucagonoma, gastrinoma, carcinoid tumors, and vipoma; cancers of the small bowel, e.g., adenocarcinoma, carcinoid tumors, Kaposi's sarcoma, leiomyoma, hemangioma, lipoma, neurofibroma, and fibroma; cancers of the large bowel, e.g., adenocarcinoma, tubular adenoma, villous adenoma, hamartoma, and leiomyoma;

genitourinary tract cancers, including, for example, cancers of the kidney, e.g., adenocarcinoma and Wilm's tumor (nephroblastoma); cancers of the bladder and urethra, e.g., squamous cell carcinoma, transitional cell carcinoma, and adenocarcinoma;

liver cancers, including, for example, hepatoma, e.g., hepatocellular carcinoma; cholangiocarcinoma; hepatoblastoma; angiosarcoma; hepatocellular adenoma; and hemangioma;

bone cancers, including, for example, osteogenic sarcoma (osteosarcoma), fibrosarcoma, malignant fibrous histiocytoma, chondrosarcoma, Ewing's sarcoma, malignant giant cell tumor chordoma, osteochondroma (osteocartilaginous exostoses), benign chondroma, chondroblastoma, chondromyxofibroma, osteoid osteoma and giant cell tumors;

nervous system cancers, including, for example, cancers of the skull, e.g., osteoma, hemangioma, granuloma, xanthoma, and osteitis deformans; cancers of the meninges, e.g., meningioma, meningiosarcoma, and gliomatosis; cancers of the brain, e.g., astrocytoma, medulloblastoma, glioma, ependymoma, germinoma (pinealoma), glioblastoma multiform, oligodendroglioma, schwannoma, retinoblastoma, and congenital tumors; and cancers of the spinal cord, e.g., neurofibroma, meningioma, glioma and sarcoma;

skin cancers, including, for example, malignant melanoma, basal cell carcinoma, squamous cell carcinoma, Kaposi's sarcoma, moles dysplastic nevi, lipoma, angioma, dermatofibroma, keloids, psoriasis; and

adrenal gland cancers, including, for example, neuroblastoma.

Cancers envisioned for prognosis only in female patients by the inventions disclosed herein include, but are not limited to, the following:
gynecological cancers, including, for example, cancers of the uterus, e.g., endometrial carcinoma; cancers of the cervix, e.g., cervical carcinoma, and pre-tumor cervical dysplasia; cancers of the ovaries, e.g., ovarian carcinoma, including serous cystadenocarcinoma, mucinous cystadenocarcinoma, unclassified carcinoma, granulose-thecal cell tumors, Sertoli-Leydig cell tumors, dysgerminoma, and malignant teratoma; cancers of the vulva, e.g., squamous cell carcinoma, intraepithelial carcinoma, adenocarcinoma, fibrosarcoma, and melanoma; cancers of the vagina, e.g., clear cell carcinoma, squamous cell carcinoma, botryoid sarcoma, and embryonal rhabdomyosarcoma; and cancers of the fallopian tubes, e.g., carcinoma.

Cancers envisioned for treatment only in male patients by the inventions disclosed herein include, but are not limited to, the following:
cancer of the testis in the male patients, e.g., seminoma, teratoma, embryonal carcinoma, teratocarcinoma, choriocarcinoma, sarcoma, interstitial cell carcinoma, fibroma, fibroadenoma, adenomatoid tumors, and lipoma.

Cancers may be solid tumors that may or may not be metastatic. The term “tumor cell,” as provided herein, includes a cell afflicted by any one of the above identified disorders.

Determination of TUBB3 or TUBB6 gene expression level may be carried out by quantifying with respect to a control the expressed polypeptide translated from the TUBB3 or TUBB6 gene transcript. In this case, a protein sample is first prepared from a biological sample, e.g. a solid tumor cell sample, and the expression of respective proteins is detected. The sample can be a tissue sample taken from a biopsy and mounted in any way known to the skilled artisan. The tumor cells can either be free tumor cells derived from a patient, or cells of a tumor cell line derived from the patient. Specimens include but are not limited to standard paraffin embedded slides normally used in standard diagnostic pathology. In some embodiments, sections of 3 μm thick or more are prepared. The sections are mounted onto poly-L-lysine coated slides. The slides are dried at 37° C. overnight and rehydrated with xylene before blocking of the endogenous peroxidase with 3% H₂O₂ in distilled water for 5 minutes. Non specific binding is reduced through incubation with 20% normal rabbit serum for 25 minutes at room temperature. The detection described can be carried out with any protein detection method known in the art allowing to count the cells expressing the desired protein and the cells not expressing the desired protein, or allowing in any manner possible to establish a percentage of cells expressing the protein in the sample analyzed. This includes but is not limited to visual inspection and microscopic analysis as well as automatic image analysis performed with dedicated software on the images obtained by microscopy. The detection shall enable the person carrying out the method described herein, to assess directly (by direct count) or indirectly (by calculating an average of cells per area and assessing the percentage of the area in which the protein of interest can be detected) a percentage of tumor cells expressing the protein.

Any methods available in the art for detecting and quantifying polypeptide encoded by a TUBB3 or TUBB6 gene according to the invention, is encompassed. Such methods may rely on utilizing a substance comprising a binding moiety for the polypeptide. Assays based on TUBB3 or TUBB6 protein-specific biomolecule interaction include, but are not limited to, antibody-based assays, aptamer-based assays, receptor and ligand assays, enzyme activity assays, and allosteric regulator binding assays. The invention is not limited to any one method of protein quantification with respect to a control recited herein, but rather encompasses all presently known or heretofore unknown methods, such as methods that are discovered in the art. Proteins may be detected by other methods, e.g., mass spectroscopy analysis, that do not rely on a binding moiety.

In one embodiment, the substance comprises an antibody that specifically binds to TUBB3 or TUBB6 protein. Antibodies can be used in various immunoassay-based protein determination methods such as Western blot analysis, immunoprecipitation, radioimmunoassay (RIA), immunofluorescent assay, chemiluminescent assay, flow cytometry, immunocytochemistry, immunohistochemistry and enzyme-linked immunosorbent assay (ELISA).

In immunohistochemistry, the count is made, by way of example, by counting the stained cells expressing TUBB3 protein or TUBB6 protein, or by calculating through image analysis and a dedicated software the threshold above which a given signal is positive and then the area percentage, in a given image, exhibiting a signal above the threshold. In general, in an “immunohistochemical method” a section of tissue is tested, by way of example, by exposing the tissues to antibodies that are specific for the protein that is being assessed. The antibodies are then visualized by any of a number of methods to determine the presence and the amount of the protein present. Examples of commonly known methods that may be used to visualize antibodies include but are not limited to the use of luciferase, alkaline phosphatase, horseradish peroxidase or P-galactosidase or chemical methods such as DAB/Substrate chromogen, gold, fluorescent or labeled antibodies and by any of the many different methods known to those skilled in the art.

In an embodiment of the immunohistochemical method (and the kit for carrying out the method) according to the present description and claims, combined detection or assaying of the expression level of TUBB3 protein or TUBB6 protein percentage of expression in a tumor sample or in cancer cells includes contacting the samples or the cells with an antibody, or with an antigen-binding fragment thereof specific for TUBB3 protein or TUBB6 protein, or a fragment thereof, and determining the amount of the binding antibody on the tumor, or on the cancer cells. Two consecutive sections can be used or alternatively the same section can be used with a dual labeling immunohistochemical method, in which revelation of the protein is performed using two diverse techniques yielding at the end as output two different colors, in a way that in the same sample both antigens can be individually assessed. In both cases, the antibody can be labeled by any detectable means, which includes but is not limited to enzymatically, radioactively, fluorescently, chemiluminescently or bioluminescently labeled antibodies by any of the many different methods known to those skilled in this art. By combined detection of “active fragments thereof” fragments such as Fv, Fab, F(ab′)₂, scFv, ScFv-Fc, specifically binding both TUBB3 protein and TUBB6 protein on the same specimen are included.

In an enzyme-linked immunosorbent assay (ELISA), an enzyme such as, but not limited to, horseradish peroxidase (HRP), alkaline phosphatase (AP), beta-galactosidase or urease can be linked, for example, to an antigen antibody or to a secondary antibody for use in a method of the invention. A horseradish-peroxidase detection system may be used, for example, with the chromogenic substrate tetramethylbenzidine (TMB), which yields a soluble product in the presence of hydrogen peroxide that is detectable at 450 nm. Other convenient enzyme-linked systems include, for example, the alkaline phosphatase detection system, which may be used with the chromogenic substrate p-nitrophenyl phosphate to yield a soluble product readily detectable at 405 nm. Similarly, a beta-galactosidase detection system may be used with the chromogenic substrate o-nitrophenyl-beta-D-galactopyranoside (ONPG) to yield a soluble product detectable at 410 nm. Alternatively, a urease detection system may be used with a substrate such as urea-bromocresol purple (Sigma Immunochemicals, St. Louis, Mo.). Useful enzyme-linked primary and secondary antibodies can be obtained from any number of commercial sources.

For chemiluminescence and fluorescence assays, chemiluminescent and fluorescent secondary antibodies may be obtained from any number of commercial sources. Fluorescent detection is also useful for detecting antigen or for determining a level of antigen in a method of the invention. Useful fluorochromes include, but are not limited to, DAPI, fluorescein, Hoechst 33258, R-phycocyanin, B-phycoerythrin, R-phycoerythrin, rhodamine, Texas red and lissamine- Fluorescein- or rhodamine-labeled antigen-specific antibodies.

Radioimmunoassays (RIAs) are described for example in Brophy et al. (1990, Biochem. Biophys. Res. Comm. 167:898-903) and Guechot et al. (1996, Clin. Chem. 42:558-563). Radioimmunoassays are performed, for example, using Iodine-125-labeled primary or secondary antibody.

Quantitative Western blotting may also be used to determine the level of TUBB3 or TUBB6 protein according to the present invention. Western blots are quantified using well known methods such as scanning densitometry (Parra et al., 1998, J. Vasc. Surg. 28:669-675).

A signal emitted from a detectable antibody is analyzed, for example, using a spectrophotometer to detect color from a chromogenic substrate; a radiation counter to detect radiation, such as a gamma counter for detection of Iodine-125; or a fluorometer to detect fluorescence in the presence of light of a certain wavelength. Where an enzyme-linked assay is used, quantitative analysis of the amount of antigen is performed using a spectrophotometer. It is understood that the assays of the invention can be performed manually or, if desired, can be automated and that the signal emitted from multiple samples can be detected simultaneously in many systems available commercially.

The antibody used to determine the level of TUBB3 protein or TUBB6 protein in a sample in an immunnoassay can comprise a polyclonal or monoclonal antibody. The antibody can comprise an intact antibody, or antibody fragments capable of specifically binding TUBB3 protein or TUBB6 protein. Such fragments include, but are not limited to, Fab and F(ab')₂ fragments.

When the antibody used in the methods of the invention is a polyclonal antibody (IgG), the antibody is generated by inoculating a suitable animal with a TUBB3 protein or TUBB6 protein, peptide or a fragment thereof. Antibodies produced in the inoculated animal which specifically bind TUBB3 protein or TUBB6 protein are then isolated from fluid obtained from the animal. Antibodies may be generated in this manner in several non-human mammals such as, but not limited to goat, sheep, horse, rabbit, and donkey. Methods for generating polyclonal antibodies are well known in the art and are described, for example in Harlow, et al. (1988, In: Antibodies, A Laboratory Manual, Cold Spring Harbor, N.Y.). These methods are not repeated herein as they are commonly used in the art of antibody technology.

When the antibody used in the methods of the invention is a monoclonal antibody, the antibody is generated using any well known monoclonal antibody preparation procedures such as those described, for example, in Harlow et al. (supra) and in Tuszynski et al. (1988), Blood, 72:109-115. Given that these methods are well known in the art, they are not replicated herein. Generally, monoclonal antibodies directed against a desired antigen are generated from mice immunized with the antigen using standard procedures as referenced herein. Monoclonal antibodies directed against full length or peptide fragments of a TUBB3 protein or TUBB6 protein may be prepared using the techniques described in Harlow, et al., supra.

Techniques for detecting and quantifying (such as with respect to a control) antibody binding are well-known in the art. Antibody binding to a TUBB3 protein or TUBB6 protein may be detected through the use of chemical reagents that generate a detectable signal that corresponds to the level of antibody binding and, accordingly, to the level of TUBB3 or TUBB6 protein expression. Examples of such detectable substances include enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include ¹²⁵I,¹³¹I, ³⁵S, or ³H.

Antibody binding may be detected through the use of a secondary antibody that is conjugated to a detectable label. Examples of detectable labels include but are not limited to polymer-enzyme conjugates. The enzymes in these complexes are typically used to catalyze the deposition of a chromogen at the antigen-antibody binding site, thereby resulting in cell staining that corresponds to expression level of the protein of interest (in this case TUBB3 or TUBB6). Preferred enzymes of particular interest include horseradish peroxidase (HRP) and alkaline phosphatase (AP).

A protein assay may be employed that combines antibody-protein binding with detection of the reporter nucleic acid by real-time PCR, e.g., TaqMan® Chemistry-Based Protein Assay, Applied BioSystems by Life Technologies Corporation, Carlsbad, Calif. The latter is a proximity ligation assay based upon Fredriksson et al., Nat. Biotechnol. 2002, 20:473-477 and Gullberg et al., Proc Natl Acad Sci USA. 2004, 101(22):8420-4.

TUBB3 or TUBB6 proteins can be detected and quantified by aptamer-based assays, which are very similar to antibody-based assays, but with the use of an aptamer instead of an antibody. An “aptamer-based” assay is thus an assay for the determination of polypeptide that relies on specific binding of an aptamer. An aptamer can be any polynucleotide, generally a RNA or a DNA, which has a useful biological activity in terms of biochemical activity or molecular recognition attributes. Usually, an aptamer has a molecular activity such as having an enzymatic activity or binding to a polypeptide at a specific region (i.e., similar to an epitope for an antibody) of the polypeptide. It is generally known in the art that an aptamer can be made by in vitro selection methods. In vitro selection methods include a well known method called systematic evolution of ligands by exponential enrichment (a.k.a. SELEX). Briefly, in vitro selection involves screening a pool of random polynucleotides for a particular polynucleotide that binds to a biomolecule, such as a polypeptide, or has a particular activity that is selectable. Generally, the particular polynucleotide represents a very small fraction of the pool, therefore, a round of amplification, usually via polymerase chain reaction, is employed to increase the representation of potentially useful aptamers. Successive rounds of selection and amplification are employed to exponentially increase the abundance of a particular aptamer. In vitro selection is described in Famulok, M.; Szostak, J. W., In Vitro Selection of Specific Ligand Binding Nucleic Acids, Angew. Chem. 1992, 104:1001. (Angew. Chem. Int. Ed. Engl. 1992, 31:979-988.); Famulok, M.; Szostak, J. W., Selection of Functional RNA and DNA Molecules from Randomized Sequences, Nucleic Acids and Molecular Biology, Vol 7, F. Eckstein, D. M. J. Lilley, Eds., Springer Verlag, Berlin, 1993, pp. 271; Klug, S.; Famulok, M., All you wanted to know about SELEX; Mol. Biol. Reports 1994, 20:97-107; and Burgstaller, P.; Famulok, M. Synthetic ribozymes and the first deoxyribozyme; Angew. Chem. 1995, 107:1303-1306 (Angew. Chem. Int. Ed. Engl. 1995, 34:1189-1192), U.S. Pat. No. 6,287,765, U.S. Pat. No. 6,180,348, U.S. Pat. No. 6,001,570, U.S. Pat. No. 5,861,588, U.S. Pat. No. 5,567,588, U.S. Pat. No. 5,475,096, and U.S. Pat. No. 5,270,163, which are incorporated herein by reference.

Substantially pure TUBB3 or TUBB6, which can be used as an immunogen for raising polyclonal or monoclonal antibodies, or as a substrate for selecting aptamers, can be prepared, for example, by recombinant DNA methods. For example, the cDNA of the TUBB3 or TUBB6 protein can be cloned into an expression vector by techniques within the skill in the art. An expression vector comprising sequences encoding the maker protein can then be transfected into an appropriate, for example bacterial, host, whereupon the protein is expressed. The expressed protein can then be isolated by any suitable technique.

For example, TUBB3 or TUBB6 can be prepared in the form of a bacterially expressed glutathione S-transferase (GST) fusion protein. Such fusion proteins can be prepared using commercially available expression systems, following standard expression protocols, e.g., “Expression and Purification of Glutathione-S-Transferase Fusion Proteins”, Supplement 10, unit 16.7, in Current Protocols in Molecular Biology (1990) and Smith and Johnson, Gene 1988, 67:34-40; Frangioni and Neel, Anal. Biochem. 1993, 210:179-187, the entire disclosures of which are herein incorporated by reference.

The practice of the invention is readily adapted to kit form. The identification of the link between TUBB3 and TUBB6 gene expression and prognosis of cancer in a female patient with a solid tumor provides the basis for clinical diagnostic kits based on protein expression.

Basic materials and reagents required for determination of prognosis of cancer in a female patient with a solid tumor according to the invention may be assembled in a kit. Provided is a kit for determining the prognosis of cancer in a female patient with a solid tumor comprising a set of reagents that specifically detects the protein expression levels of TUBB3 and TUBB6 in cells from the solid tumor, and instructions for using the kit for evaluating the prognosis of cancer in a female patient with a solid tumor. In certain embodiments, the kit comprises at least one reagent that specifically detects protein expression levels of TUBB3 and TUBB6, and instructions for using the kit according to one or more methods of the invention. Each kit necessarily comprises reagents which render the procedure specific. For example, for each of TUBB3 protein or TUBB6 protein, the kit can comprise an antibody, an antibody derivative, or an antibody fragment that binds specifically with TUBB3 protein or TUBB6 protein.

Depending on the procedure, the kit may further comprise one or more of: immunodetection buffer and/or reagents, labeling buffer and/or reagents, and detection means. Protocols for using these buffers and reagents for performing different steps of the procedure may also be included in the kit.

Reagents may be supplied in a solid (e.g., lyophilized) or liquid form. Kits of the present invention may optionally comprise different containers (e.g., vial, ampoule, test tube, flask or bottle) for each individual buffer and/or reagent. Each component will generally be suitable as aliquoted in its respective container or provided in a concentrated form. Other containers suitable for conducting certain steps for the disclosed methods may also be provided. In certain embodiments, the kits of the present invention further comprise control samples.

Instructions for using the kit according to one or more methods of the invention may comprise instructions for processing the solid tumor cell samples, and/or performing the test, and instructions for interpreting the results.

The kit would include instructional material that informs the user of the relationship between expression level and cancer prognosis in a female patient with a solid tumor, such as a package insert comprising the explanation that detection of TUBB6 protein expression in a percentage of solid tumor cells of the female patient that is less than the median expression for TUBB6 protein and detection of TUBB3 protein expression in a percentage of solid tumor cells of the female patient that is less than the median percentage expression for TUBB3 protein indicates a patient with a good prognosis, whereas the detection of TUBB6 protein expression in a percentage of solid tumor cells of the female patient that is equal to or greater than the median percentage expression for TUBB6 protein and/or detection of TUBB3 protein expression in a percentage of solid tumor cells of the female patient that is equal to or greater than the median percentage expression for TUBB3 protein indicates a patient with a poor prognosis. The instructional material may comprise a publication, a recording, a diagram, or any other medium of expression which can be used to communicate the usefulness of the method of the invention in the kit for assessment of cancer prognosis in a female patient with a solid tumor. The package insert may comprise text housed in any physical medium, e.g., paper, cardboard, film, or may be housed in an electronic medium such as a diskette, chip, memory stick or other electronic storage form. The instructional material of the kit of the invention may, for example, be affixed to a container which contains other contents of the kit, or be shipped together with a container which contains the kit. Alternatively, the instructional material may be shipped separately from the container with the intention that the instructional material and the contents of the kit be used cooperatively by the recipient.

For antibody-based kits, the kit can comprise, for example: (1) a first antibody (e.g., attached to a solid support) that binds to TUBB3 protein or TUBB6 protein; and, optionally, (2) a second, different antibody that binds to either the protein or the first antibody and is conjugated to a detectable label. The kit can further comprise components necessary for detecting the detectable label (e.g., an enzyme or a substrate), and instrumentation for detection and measurement. In some embodiments, the TUBB3 protein and TUBB6 proteins are labeled with different colors. A kit may further optionally contain aliquots of known amounts of TUBB3 protein or TUBB6 protein to serve as reference standards, or reference samples representing TUBB3 protein or TUBB6 protein from certain specific solid tumors.

The antibody-based kit for determination of TUBB3 protein or TUBB6 protein expression may further contain a package insert that informs the user of the relationship between expression level and disease prognosis, such as a package insert comprising the explanation that detection of TUBB6 protein expression in a percentage of solid tumor cells of the female patient that is less than the median expression for TUBB6 protein and detection of TUBB3 protein expression in a percentage of solid tumor cells of the female patient that is less than the median percentage expression for TUBB3 protein indicates a patient with a good prognosis, whereas the detection of TUBB6 protein expression in a percentage of solid tumor cells of the female patient that is equal to or greater than the median percentage expression for TUBB6 protein and/or detection of TUBB3 protein expression in a percentage of solid tumor cells of the female patient that is equal to or greater than the median percentage expression for TUBB3 protein indicates a patient with a poor prognosis.

### Methods of Treatment for Female Patients

Provided is a method for treating a female patient with a solid tumor comprising administering one or more chemotherapeutic agents to a female patient following prognosis according to the method of the first embodiment. The chemotherapeutic agent may be administered by any route, including oral, rectal, pulmonary, sublingual, and parenteral administration. Parenteral administration includes, for example, intravenous, intramuscular, intraarterial, intravesical (e.g., to the bladder), intradermal, transdermal, topical or subcutaneous administration.

Treatment with at least one chemotherapeutic agent may be given at least once per day, typically once, twice, three times or four times per day with the doses given at equal intervals throughout the day and night in order to maintain a constant presence of the drug. However, the skilled artisan will be aware that a treatment schedule can be optimized for any given patient, and that administration of compound may occur less frequently than once per day.

One or more chemotherapeutic agents may be administered simultaneously, by the same or different routes, or at different times during treatment. In preferred embodiments, the chemotherapeutic agent is oxaliplatin or irinotecan.

The treatment may be carried out for as long a period as necessary. Typically it is contemplated that treatment would be continued indefinitely while the disease state persists, although discontinuation might be indicated if the compounds no longer produce a beneficial effect. The treating physician will know how to increase, decrease, or interrupt treatment based on patient response.

The specific dose of the one or more chemotherapeutic agents to obtain therapeutic benefit for treatment of a cellular proliferative disorder will, of course, be determined by the particular circumstances of the individual patient including the size, weight, age and sex of the patient, the nature and stage of the disease, the aggressiveness of the disease, and the route of administration of the compound.

### Methods of Treatment for Male Patients

Provided is a method of sensitizing a cancer cell of a solid tumor of a male patient, other than a prostate cancer cell, to treatment with a chemotherapeutic agent comprising administering to the patient an effective amount of

(a)

(i) at least one antiandrogen;

(ii) at least one inhibitor of androgen synthesis; or

(iii) a combination of at least one antiandrogen and at least one inhibitor of androgen synthesis; and

(b) at least one chemotherapeutic agent other than an antiandrogen or an inhibitor of androgen synthesis. The chemotherapeutic agent and the antiandrogen and/or the inhibitor of androgen synthesis may be administered by any route, including oral, rectal, pulmonary, sublingual, and parenteral administration. Parenteral administration includes, for example, intravenous, intramuscular, intraarterial, intravesical (e.g., to the bladder), intradermal, transdermal, topical or subcutaneous administration.

Treatment with at least one antiandrogen and/or with at least one inhibitor of androgen synthesis may be given at least once per day, typically once, twice, three times or four times per day with the doses given at equal intervals throughout the day and night in order to maintain a constant presence of the drug in order to sufficiently sensitize the cancer cell to treatment with at lest one chemotherapeutic agent. Treatment with at least one chemotherapeutic agent may be given at least once per day, typically once, twice, three times or four times per day with the doses given at equal intervals throughout the day and night in order to maintain a constant presence of the drug. However, the skilled artisan will be aware that a treatment schedule can be optimized for any given patient, and that administration of compound may occur less frequently than once per day.

One or more antiandrogens, inhibitors of androgen synthesis and chemotherapeutic agents may be administered simultaneously, by the same or different routes, or at different times during treatment. In preferred embodiments the antiandrogen is bicalutamide, flutamide, nicalutamide, spironolactone, cyproterone acetate, dutasteride, bexlosteride, izonsteride, turosteride, epristeride or finasteride. In preferred embodiments the inhibitor of androgen synthesis is an inhibitor of CYP17. In further preferred embodiments, the inhibitor of CYP17 is ketoconazole, abiraterone, abiraterone acetate, VN/124-1(TOK-001) or TAK-700, or salts thereof. See FIGS. 14A-B. In some embodiments the inhibitor of androgen synthesis is an inhibitor of AKR1C3. In further embodiments, the inhibitor of AKR1C3 is an indomethacin analog. In yet further embodiments, the indomethacin analog is N-(4-chlorobenzoyl)-melatonin. (Byrns M C, Steckelbroeck S and Penning T M, An indomethacin analogue, N-(4-chlorobenzoyl)-melatonin, is a selective inhibitor of aldo-keto reductase 1C3 (type 2 3α-HSD, type 5 17β-HSD, and prostaglandin F synthase), a potential target for the treatment of hormone dependent and hormone independent malignancies, Biochem. Pharmacol., 2007, 75(2):484-493). In preferred embodiments, the chemotherapeutic agent is oxaliplatin or irinotecan. The one or more antiandrogens and/or inhibitors of androgen synthesis may be administered before, simultaneously with, or after the one or more chemotherapeutic agents. The one or more antiandrogens and/or inhibitors of androgen synthesis and the one or more chemotherapeutic agents may also be prescribed to be taken in combination with other drugs used to treat proliferative disorders. When used in such combinations the one or more antiandrogens, the one or more inhibitors of androgen synthesis, the one or more chemotherapeutic agents and the other drugs may be administered simultaneously, by the same or different routes, or at different times during treatment. The dose of the other drug selected will depend on the particular one or more antiandrogens and/or one or more inhibitors of androgen synthesis and one or more chemotherapeutic agents being used and the route and frequency of administration.

The treatment may be carried out for as long a period as necessary. Typically it is contemplated that treatment would be continued indefinitely while the disease state persists, although discontinuation might be indicated if the compounds no longer produce a beneficial effect. The treating physician will know how to increase, decrease, or interrupt treatment based on patient response.

The specific dose of the one or more antiandrogens and/or one or more inhibitors of androgen synthesis and the one or more chemotherapeutic agents to obtain therapeutic benefit for treatment of a cellular proliferative disorder will, of course, be determined by the particular circumstances of the individual patient including the size, weight, age and sex of the patient, the nature and stage of the disease, the aggressiveness of the disease, and the route of administration of the compound.

Also provided is a pharmaceutical pack or kit comprising one or more containers filled with the one or more antiandrogens and/or one or more inhibitors of androgen synthesis and the one or more chemotherapeutic agents. Optionally associated with such container(s) is a notice in the form prescribed by a governmental agency regulating the manufacture, use or sale of pharmaceuticals or biological products, which notice reflects approval by the agency of manufacture, use or sale for human administration.

The practice of the invention is illustrated by the following non-limiting examples. The invention should not be construed to be limited solely to the compositions and methods described herein, but should be construed to include other compositions and methods as well. One of skill in the art will know that other compositions and methods are available to perform the procedures described herein.

### EXAMPLES

### Example 1

### Predictive Value of TUBB3/TUBB6 Immunostaining

### Patients

A study included 180 colorectal cancer patients. For 147 patients, the full clinical history was available and the median follow up for them was three years. Analysis of the expression of TUBB3 and TUBB6 was performed in primary tumors from all 180 patients, resected at the first surgery before the patients had undergone any treatment for the disease. Clinical data were collected from an archive.

### Immunohistochemistry

Tumor tissues biopsies were obtained during the first surgery in all cases. Tissue specimens were fixed in 10% formalin and paraffin embedded according to standard procedures. Immunostaining was done on 3 μm tissue sections mounted on poly-l-lysine-coated slides and dried at 37° C. overnight. After the slides were deparaffinized in xylene and rehydrated conventionally, the endogenous peroxidase activity was blocked with 3% H₂O₂ in TBS for 5 minutes. Antigen retrieval procedure was done by microwave oven heating in 1 mmol/L EDTA (pH 8). Sections were incubated with 20% normal goat serum 20% for 30 minutes at room temperature to reduce nonspecific binding, then with the monoclonal anti-TUBB3 Tuj1 antibody (diluted 1:350; Covance, Princeton, N.J.) in 1% bovine serum albumin-PBS or with the polyclonal anti-TUBB6 (diluted 1:750) in house developed and previously validated (Mozzetti S, Iantomasi R, De Maria I, Prislei S, Mariani M, Camperchioli A, Bartollino S, Gallo D, Scambia G, Ferlini C. Molecular mechanisms of patupilone resistance. Cancer Res 2008, 68:10197-10204). TUBB3 and TUBB6 detection were evaluated by a labeled polymer. The EnVision-rabbit+ System-HRP System (DAKO, Carpinteria, Calif.) was used. Diaminobenzidine was used as a chromogen (DAB Substrate System, DAKO). Negative controls were done by omitting the primary antibody. Positive control for TUBB3 was represented by sections taken from the brain. The internal control was obtained through the staining of the neural structures present in the sections. For TUBB6 the internal control was represented by endothelial cells. Results were expressed as the proportion of immunostained tumor cells. The analysis of all tissue sections was done without any prior knowledge of the clinical variables by a certified pathologist by means of light microscopy. The proportion of immunostained tumor cells was scored at low magnification (5× objective lens) by evaluating the entire tumor area. The value of 35% and 60% immunostained tumor cells (corresponding to the median values) for TUBB3 and TUBB6, respectively was arbitrarily chosen as cutoff value to distinguish cases with high versus low f3 TUBB3/TUBB6 content without any prior knowledge of the clinicopathologic variables and patient clinical outcome or prognosis.

### Statistical Analysis

Fisher's exact test (or χ² test for proportion) and multivariate logistic analysis were used to analyze the distribution of TUBB3/TUBB6 positivity according to clinicopathologic features. Overall survival was calculated from the date of diagnosis to the date of progression/death or date last seen. Medians and life tables were computed using the product-limit estimate by the Kaplan and Meier method and the log-rank test was employed to assess the statistical significance. Statistical analysis was carried out using JMP (SAS). Multivariate analysis assessing the clinical role of βIII tubulin expression matched with other clinicopathologic characteristics was done by Cox proportional hazards model.

The study reported herein is an archive study which included 180 colorectal cancer patients. Main features of the patient population are reported in Table 1:

TABLE 1
Distribution of clinico-pathological characteristics
in the study population.

| Characteristics | No. pts (%) | |
|---|---|---|
| All cases | 180 | |
| Age, yrs | | |
| Median | 68 | |
| AJCC Stage | | |
| I-II | 92 | (51.1) |
| IIII-IV | 82 | (45.6.) |
| Not reported | 4 | (2.2) |
| Gender | | |
| Male | 99 | (55.0) |
| Female | 81 | (45.0) |
| Histotype | | |
| Adenocarcinoma | 156 | (86.7) |
| Mucinous | 18 | (13.3) |
| Grade | | |
| G1-2 | 126 | (70.0) |
| G3 | 48 | (26.7) |
| N/A | 6 | (3.3) |

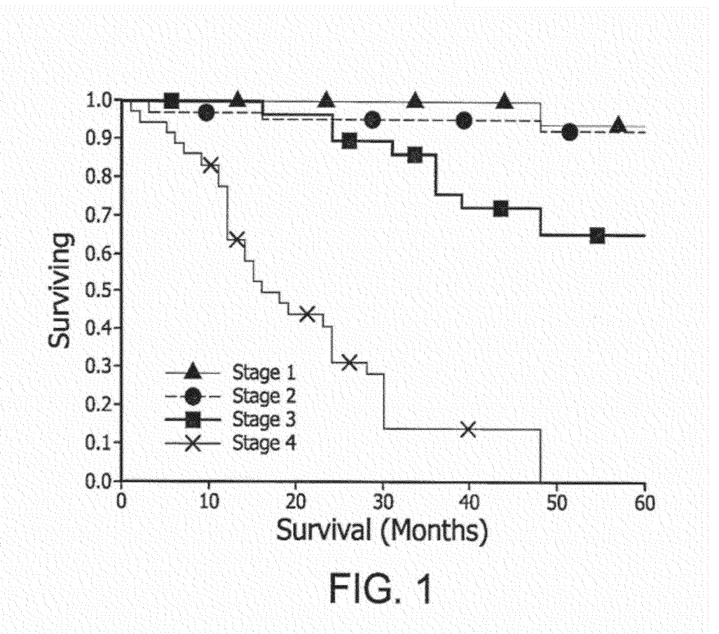

Individual slides obtained from each patient were stained with anti-TUBB3 (Tuj1) monoclonal antibody and a polyclonal anti-TUBB6 in house generated and validated as previously reported (Mozzetti S, Iantomasi R, De Maria I, Prislei S, Mariani M, Camperchioli A, Bartollino S, Gallo D, Scambia G, Ferlini C. Molecular mechanisms of patupilone resistance. Cancer Res 2008, 68:10197-10204). Regarding TUBB3 and TUBB6 immunostaining, median expression value for the two antigens was 35% and 60% for TUBB3 and TUBB6, respectively. All the specimens were collected during the first surgery and follow up data were available for 147 patients. Median follow up was 36 months and mortality rate was different by gender (39% (32/82) and 20% (13/65), for males and females, respectively). The stage of the tumor was a major determinant of the outcome as shown in FIG. 1. In this clinical setting, the risk of death at 5 years is 100% for stage 4, 32% for stage 3, and 5% for stage 1 and 2. According to TUBB3 and TUBB6 immunostaining, four groups were categorized.

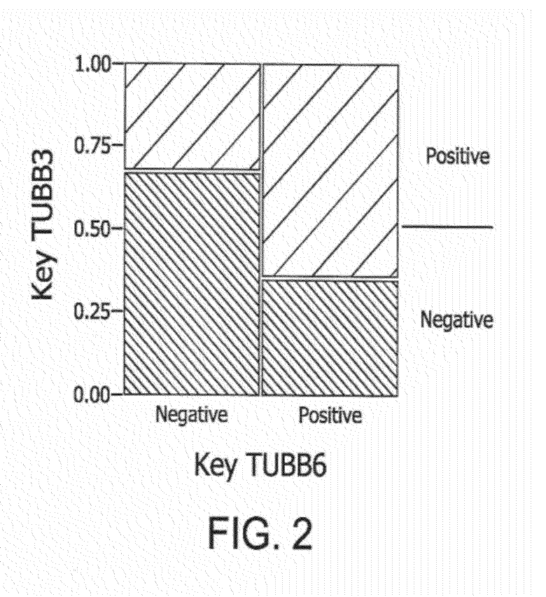

As shown in FIG. 2, groups 1, 2, 3 and 4 were double negative, single TUBB3+, single TUBB6+ and double positive, respectively, for TUBB3/TUBB6 expression. There was a tendency to co-express the two antigens with 60 and 59 patients belonging to group 1 and 4 respectively, therefore making statistically significant the probability of co-expression (See FIG. 2, Pearson's χ²=18.7, p<0.001).

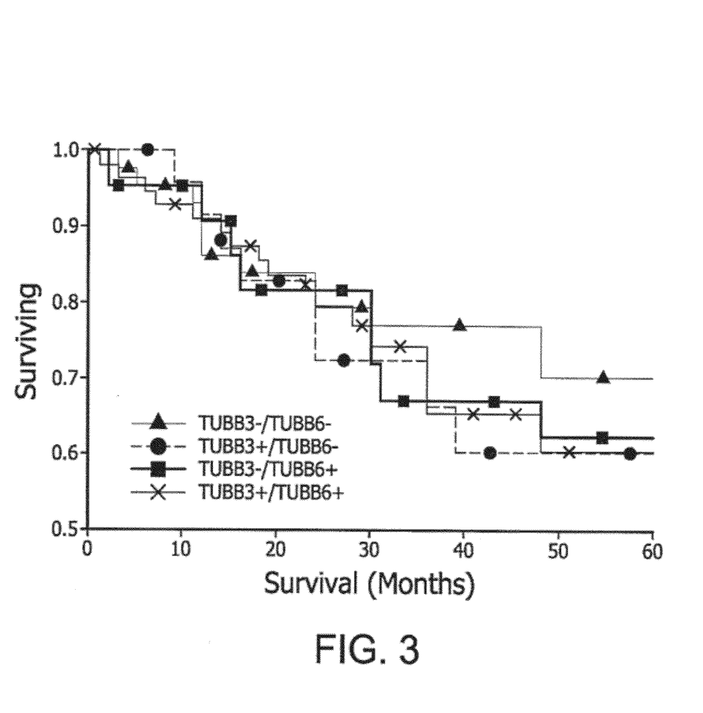

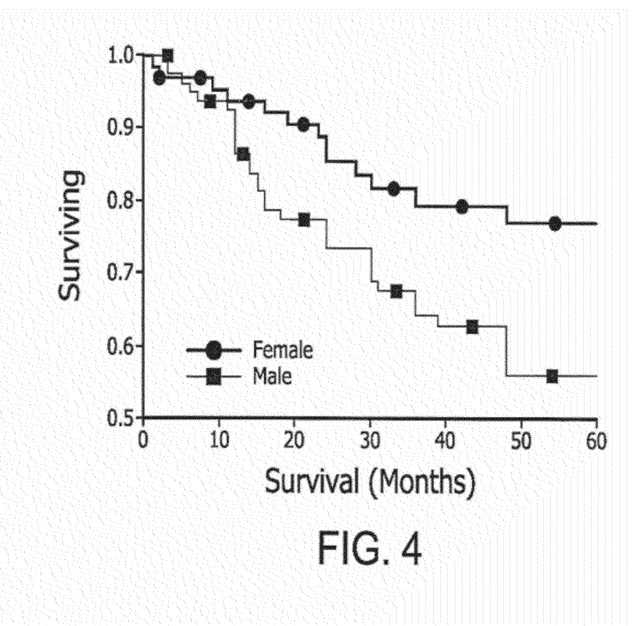

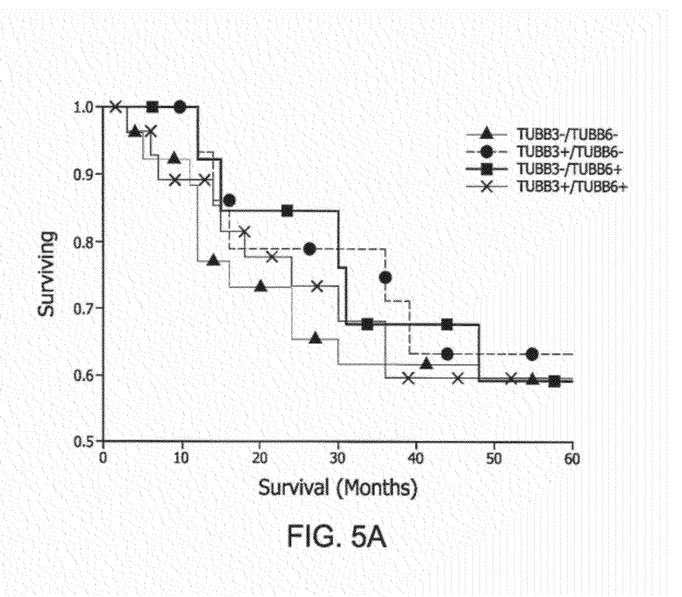

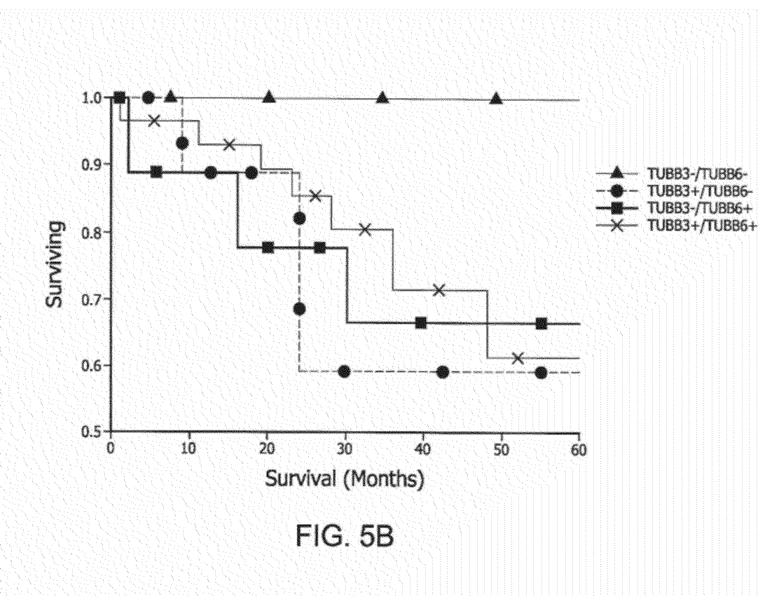

Kaplan-Meier curves for all the four groups are reported in FIG. 3 and no statistically significant differences were appreciated in the whole patient population between the four groups. When stratified for gender (FIG. 4), survival analysis revealed a statistically significant (p=0.0188) difference by gender, with the males exhibiting a poorer and more rapid outcome. When combining the two stratifications (by gender and by TUBB3/TUBB6 grouping) the behavior of the curves in males was similar to that observed in the general population without any statistically significant change between the four groups (FIG. 5A). On the other hand, in females, double negative patients (n=18) performed very well and all survived, while mortality in the remaining three subsets was about 28% (13/47) (FIG. 5B). The difference in behavior between double negative patients as compared with the three other groups was statistically significant in females (p value =0.022) while the other differences were not.

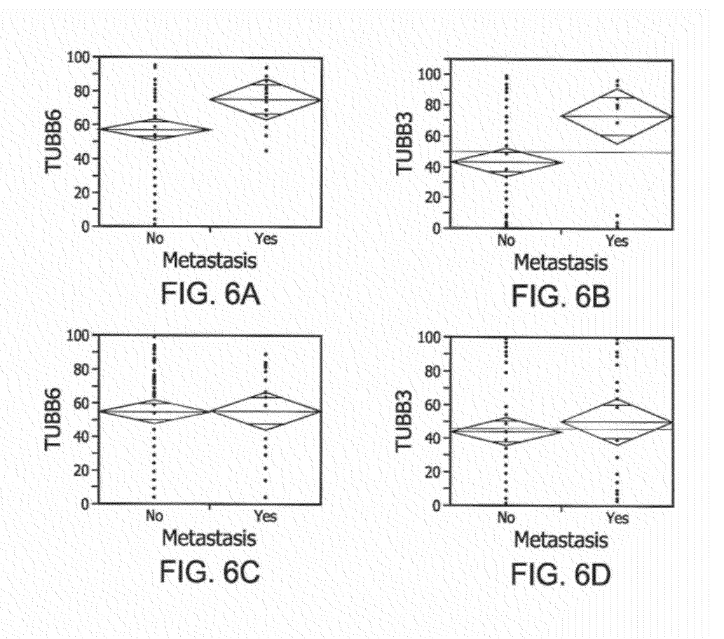

A logistic analysis was performed to identify factors correlated to the expression of TUBB3 and TUBB6 in both genders. A statistically significant correlation (FIG. 6) between the expression of both TUBB3 and TUBB6 and presence of metastasis was noticed in females (Wilcoxon/Kruskal Wallis test p value=0.0073 and 0.0084 for TUBB3 and TUBB6, respectively), but not in males (Wilcoxon/Kruskal Wallis test p value=0.67 and 0.73 for TUBB3 and TUBB6, respectively). This finding suggests that expression of TUBB3 and TUBB6 is associated with the formation of metastasis only in females, thereby suggesting a different regulation of expression of both TUBB3 and TUBB6 in the two genders.

### Example 2

### Gender Influences Status of TUBB3/TUBB6 Pathway Activation

A panel of 22 colorectal cancer cell lines was used to analyze the expression of both TUBB3 and TUBB6 in basal conditions and after serum starvation, as a stressor capable of activating the TUBB3 pathway. Nine cell lines came from female patients (WiDR, HT29, LS-174T, SW48, RKO, CO115, SW403, Colo320 and KM12) while 13 were from males (HCT116, SW480, SW837, SW116, SK-CO-1, DLD1, HCT15, LoVo, Colo201, Colo205, SW620, T84 and CACO2). This analysis was performed at the gene and protein level.

### Cell Cultures

Cell lines used in this study were obtained as standard tissue bank cells, such as ATCC and ECACC cells. Nine cell lines originated from female patients (WiDR, HT29, LS-174T, SW48, RKO, CO115, SW403, Colo320 and KM12) while 13 cell lines originated from males (HCT116, SW480, SW837, SW116, SK-CO-1, DLD1, HCT15, LoVo, Colo201, Colo205, SW620, T84 and CACO2). All cell lines were grown in DMEM medium supplemented with 10% FCS, 1% non- essential amino acid in standard cell incubators. For experiment of serum starvation cells were plated at standard density (75,000 cells/ml). After 24 hours the medium was replaced with standard medium without FCS. The count of cells was performed using a Burkitt chamber and a light microscope.

Drug treatment was performed in 6-well plates as follows. Cells were plated at 75,000 cells /mL and after an overnight treated with either oxaliplatin or SN38 at the indicated concentrations and then washed and kept for additional 72 hours in the presence of bicalutamide (20 μM). The controls were represented by the same cells treated with the vehicle alone (DMSO 0.2%). Thereafter cells were trypsinised and counted and 350 cells were plated on a Petri dish and cultured in full medium for 14 days. Thereafter, colonies were stained with 2% methylene blue and then imaged and counted using automatic image analysis (Carestream, Image Station 4000R PRO). The number of colonies obtained in control cells represented 100%.

### Gene and Protein Expression

Analysis of TUBB3 and TUBB6 expression was performed at the gene and protein level using qPCR and Western blot, respectively as previously described (Mozzetti S, Iantomasi R, De Maria I, Prislei S, Mariani M, Camperchioli A, Bartollino S, Gallo D, Scambia G, Ferlini C. Molecular mechanisms of patupilone resistance. Cancer Res 2008, 68:10197-10204). Expression of AR isoforms 1 and 2 was performed using qPCR and the following primers: forward 5′-CTCTTCAGCATTATTCCAGTG-3′ (SEQ ID NO: 1); reverse 5′-CGAGTTCCTTGATGTAGTTC-3′ (SEQ ID NO: 2). All the analyses were performed using the iCycler MyiQ2 Two color Real-Time PCR Detection System and the iQ Sybr Green Mix (Biorad) according to manufacturer's directions. For Western blots the same antibodies used for immunohistochemistry were employed and the 3A2 mouse monoclonal anti-HuR antibody (Santa Cruz) was used as loading control. In order to quantify the expression, densitometric analysis was performed in the bands of TUBB3, TUBB6 and the RNA-binding protein HuR. Values were normalized for the loading (HuR) and then quantified by dividing the value obtained in both TUBB3 and TUBB6 over that obtained in control cells and expressed as percent of control. Quantification of the bands was performed using the Carestream, Image Station 4000R PRO, according to manufacturer's directions. In all the experiments cell lines were grouped by gender and statistical analysis was performed using JMP and one-way analysis of variance. In experiment of serum starvation, 100% was represented by the expression values obtained in the cells kept in full medium.

### Results

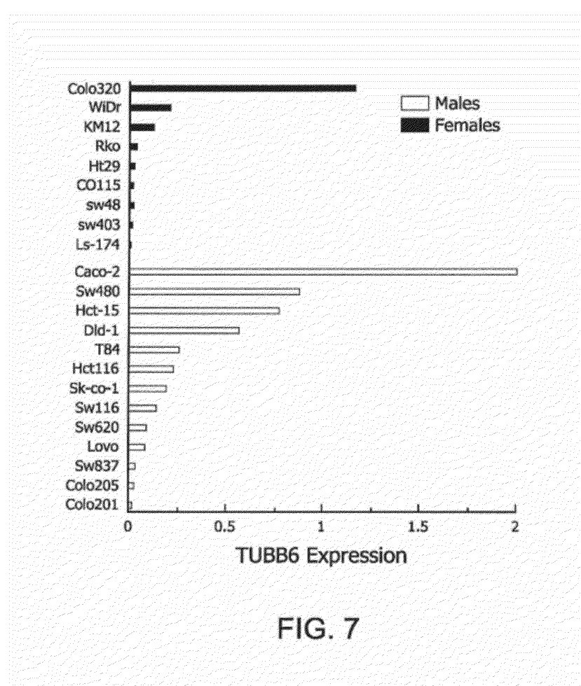

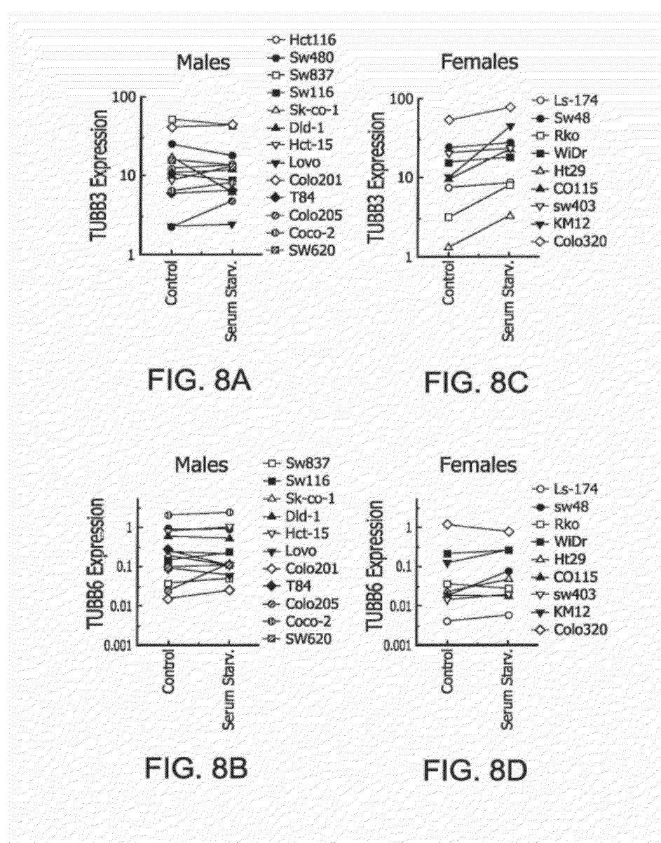

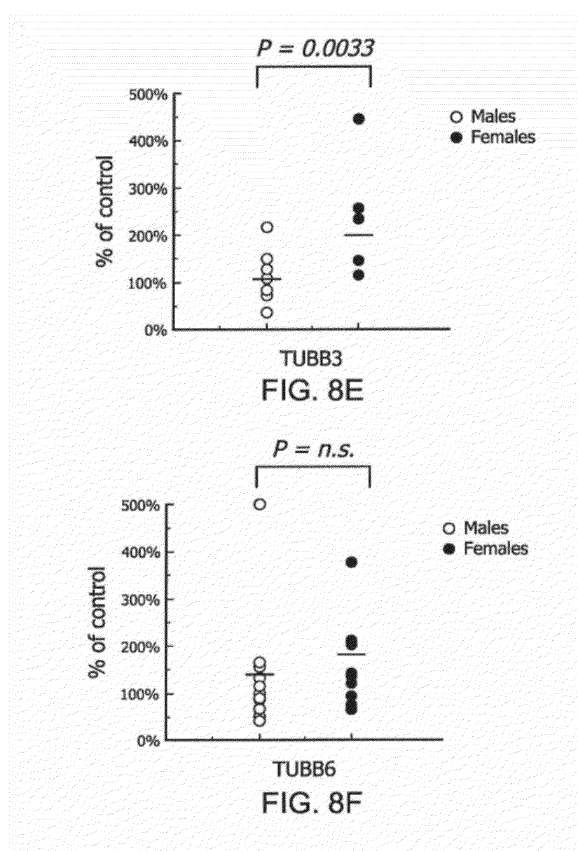

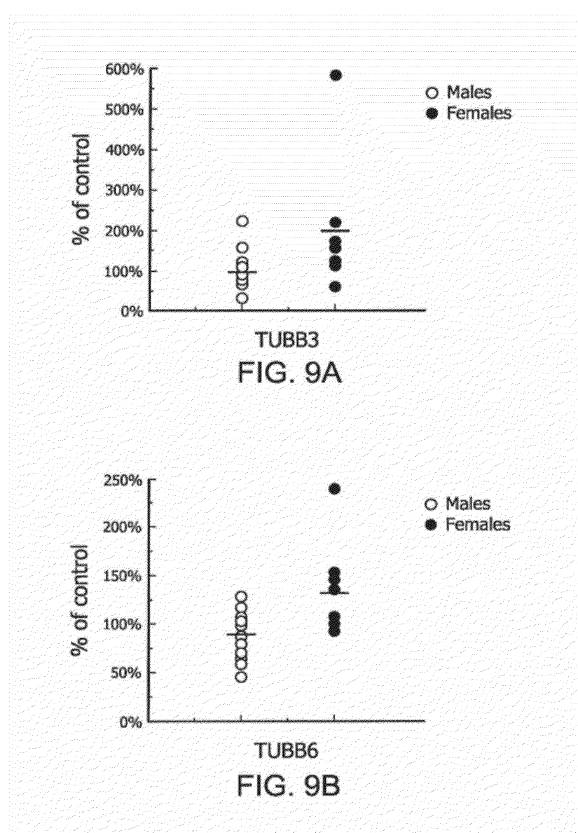

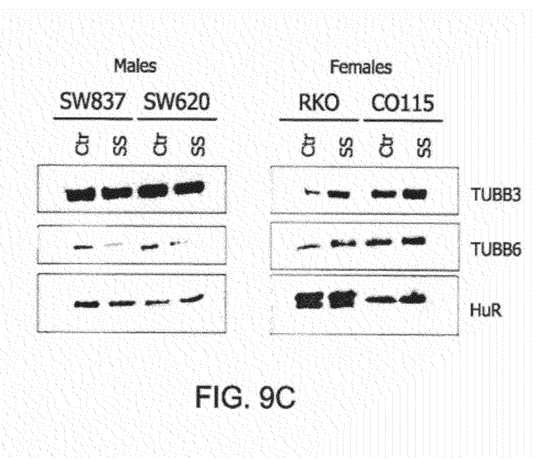

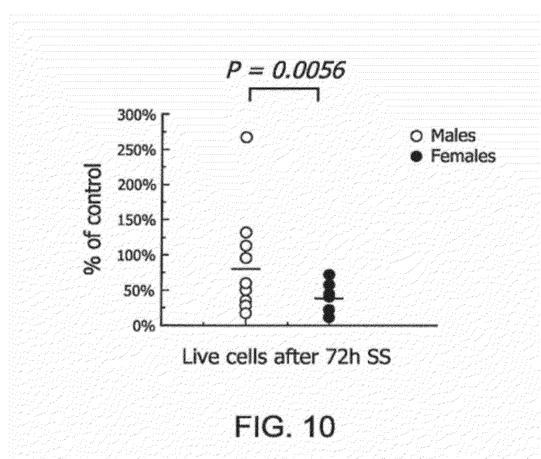

In basal conditions in females there was a statistically significant correlation between the expression of TUBB3 and TUBB6 (R=0.8806, p=0.0017). This phenomenon was not present in male cancer cell lines. When pooled together the basal expression of TUBB3 did not reveal any statistical significance between cell lines originating from males (mean 11.6) and females (11.3), while at the gene level TUBB6 expression was higher in the first (mean 0.4±0.55) than the second (mean 0.18±0.37, p=0.0488, FIG. 7). Treatment with serum starvation was able to induce in cell lines originating from females a TUBB3 increase at the gene level, while this phenomenon was not present in males (FIG. 8). As compared to the control, the mean increase of TUBB3 for female cell lines was 1.99+1.11 against 1.07+0.43 recorded in male cell lines. The difference was statistically significant (p=0.0033). TUBB6 also exhibited a tendency to increase in female cell lines upon serum starvation (mean 1.89±0.96) as compared to males (mean 1.03±0.30), but the difference was not statistically significant (p=0.139). At the protein level, the same trend was revealed (FIG. 9). TUBB3 expression significantly increased upon serum starvation in female cell lines (mean 1.95±1.54) while in males cells remained essentially stable (mean 1.09±0.42). The difference as compared to the control was statistically significant (p=0.003) only in females. At the protein level, an increased expression of TUBB6 in female cell lines was observed (mean 1.28±0.47) as compared to the male cell lines (mean 0.89±0.25), where a trend to a reduction of the protein was noticed in most cell lines. Activation of the survival pathway mirrored the sensitivity to serum starvation. In male cell lines in which the survival pathway is pre-activated (higher levels of TUBB6) inventors observed a relative resistance to serum starvation with the number of live cells (assuming 1 as control) after 72 hours having a mean value of 0.79±0.68 while in female cell lines the mean value was 0.36±0.16. Difference between genders exhibited a borderline significance (p=0.056, FIG. 10).

Altogether these findings indicate that the TUBB3/TUBB6 pathway is inducible only in cell lines originating from females. In males the pathway appears to be activated independently of serum starvation treatment, as demonstrated by constitutive and not inducible high TUBB6 expression levels and relative resistance to serum starvation. Without wishing to be limited to one theory, one possible explanation for the fact that in both patients and cancer cell lines TUBB3/TUBB6 pathway is activated is that androgens are responsible for the activation of this survival pathway.

### Example 3

### Combination of Antiandrogens With Chemotherapy in Colorectal Cancer Cell Lines

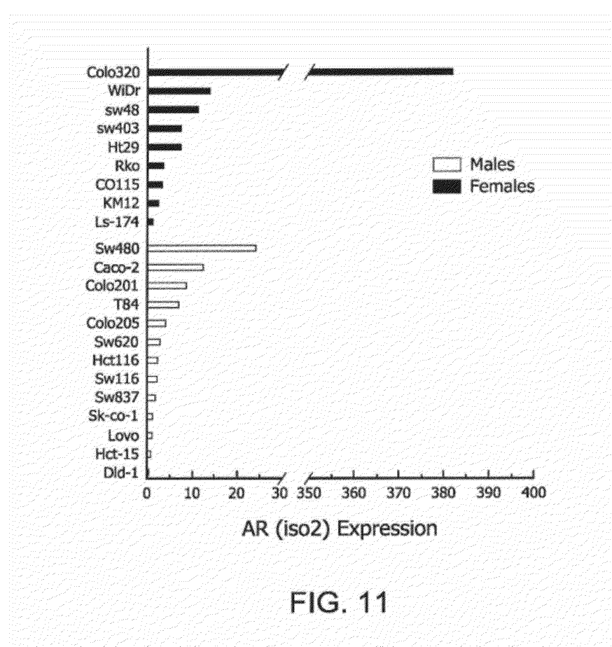

The expression of Androgen Receptor (AR, isoforms 1 and 2) was investigated in the panel of cell lines of Example 2 at the gene level using qPCR. Male cell lines with the highest expression of AR (SW480 and Caco-2) exhibited also the highest TUBB6 expression, in keeping with the hypothesis linking TUBB6 to AR. (FIG. 11). Importantly, in females, the cell line COLO-320 exhibited a dramatic upregulation of AR, and this fact explains the high expression of TUBB6 noticeable in this cell line (FIG. 11).

Oxaliplatin and irinotecan are the most important chemotherapeutics used today in the treatment of advanced colorectal cancer. The following experiment illustrates that treatment with antiandrogens can enhance efficacy of chemotherapy using those agents. A 14-day clonogenic assay of RKO (female) and SW480 (male) cells was performed with and without combination of the antiandrogen bicalutamide at a fixed concentration of 20 μM. RKO is a cell line in which in basal condition the TUBB3/TUBB6 pathway is believed to be off. The pathway is believed to be constitutively activated in SW480 cells, as is noticeable from the high levels of TUBB6. Oxaliplatin was used at four concentrations (750, 1500, 3000 and 6000 nM) and SN38 (the active metabolite of irinotecan) was used at 0.25, 0.5, 1 and 2 nM after preliminary experiments for concentration curve calibration.

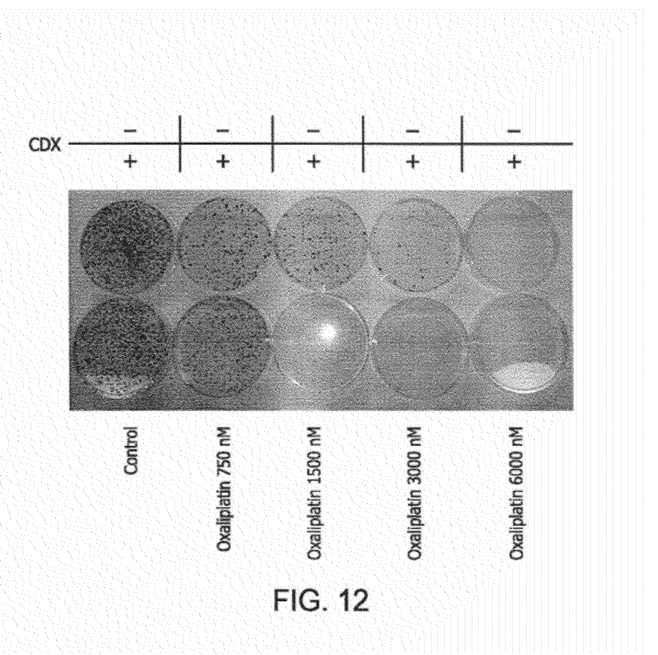

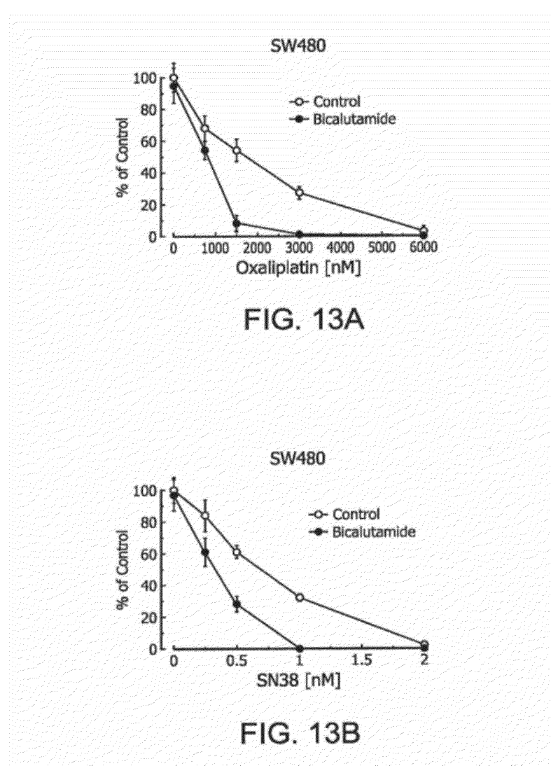

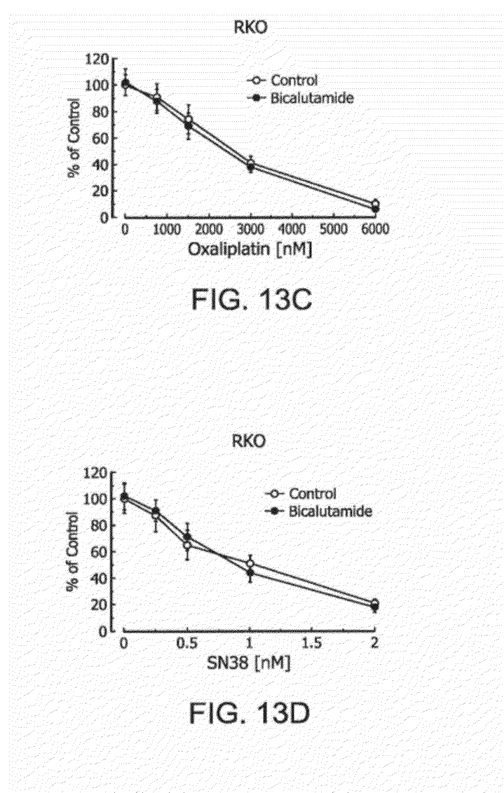

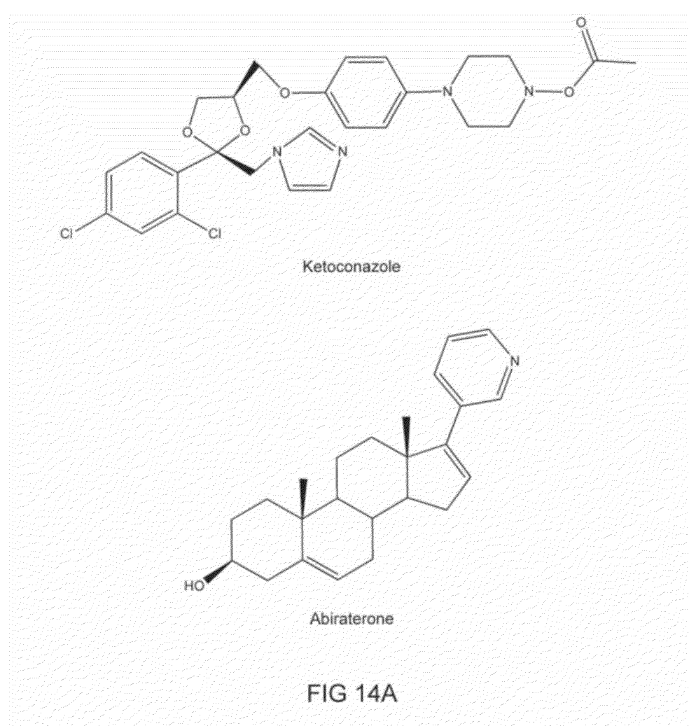

A representative experiment with oxaliplatin in SW480 cells is shown in FIG. 12. FIG. 13 represents the average of the three independent experiments in both SW480 and RKO cell lines, with both drugs. The antiandrogen bicalutamide was able to increase the efficacy of chemotherapy with both oxaliplatin and SN38 in the SW480 cell line (a male cell line which exhibits a phenotype of activated status of TUBB6 pathway) but not in RKO cells, a female cell line whose TUBB3/TUBB6 status is not constitutively active. These findings indicate that in male colorectal cancer patients treatment with antiandrogens can enhance the therapeutic effects of chemotherapy.

### Example 4

### Deleterious Effect of the G Allele in the RS743572 Single Nucleotide Polymorphism (SNP) (CYP17) in Male Patients

The effect of the RS743572 single nucleotide polymorphism (SNP) (CYP17) in male versus female colorectal cancer patients was studied. The RS743572 SNP is located in the CYP17A1 gene, which codes for the CYP17 enzyme. CYP17 is an enzyme which is capable of directing steroid synthesis towards the synthesis of androgens, and has two key enzymatic activities:

(i) the 17α-hydroxylase activity typically converts pregnenolone to 17α-hydroxypregnenolone and converts progesterone to 17α-hydroxyprogesterone; and

(ii) the C17,20-lyase activity converts 17α-hydroxypregnenolone to DHEA and 17α-hydroxyprogesterone to androstenedione.

The RS743572 SNP most represented in the population carries an A allele in the 5′-UTR of the CYP17A1 gene. The G allele has an additional binding site for the Sp-1 transcription factor, which leads to increased transcription of the gene and enhanced levels of androgens (Habuchi T, Liqing Z, Suzuki T, Sasaki R, Tsuchiya N, Tachiki H, Shimoda N, Satoh S, Sato K, Kakehi Y, Kamoto T, Ogawa O, and Kato T, Increased risk of prostate cancer and benign prostatic hyperplasia associated with a CYP17 gene polymorphism with a gene dosage effect, Cancer Res., 2000, 60:5710-5713; Lunn R M, Bell D A, Mohler L and Taylor J A, Prostate cancer risk and polymorphism in 17 hydroxylase (CYP 17) and steroid reductase (SRD5A2), Carcinogenesis, 1999, 20(9):1727-1731). The “A allele” that we are referring to corresponds to the “Al” allele in Habuchi et al. and our “G allele” corresponds to the “A2” allele of Habuchi et al.

### Materials and Methods

For DNA, formalin-fixed paraffin-embedded (FFPE) samples were obtained from patients using lymph nodes exempt from disease, or if not available, slices containing minimal or no amount of cancer cells at the pathologist's review. FFPE samples were cut to 10 μm thickness and five tissue slices were put into a 1.5 ml tube. Buffer G2 and Proteinase K were added, followed by an overnight incubation at 56° C. with shaking Genomic DNA was then automatically extracted using the EZ1 instrument with the EZ1 DNA Tissue Kit (Qiagen, Valencia, Calif.) following manufacturer's protocols. Genotyping of CYP17 (RS743572) was determined using PCR pyrosequencing with the PyroMark PCR Kit (Qiagen, Valencia, Calif.). The PCR was performed in a 25 μl mixture containing 20-40 ng of genomic DNA template, 2× PyroMark Master Mix, 10× CoralLoad, 10× Primer Set. The PCR began with a denaturation step at 95° C. for 15 minutes, followed by 45 cycles of denaturing at 94° C. for 30 seconds, annealing at 60° C. for 30 seconds, plus extension at 72° C. for 30 seconds, and ended with a final elongation step at 72° C. for 10 minutes. We used the following primers: for CYP17 (RS743572) forward primer 5′-CGGCAGGCAAGATAGACAG-3′ (SEQ ID NO: 3) and reverse primer 5′-biotin-TGGGCTCCAGGAGAATCTTT-3′ (SEQ ID NO: 4). Pyrosequencing of 20 μl of the PCR product immobilized onto the beads was performed using the following sequencing primer: 5′-CAGGCAAGATAGACAGC-3′ (SEQ ID NO: 5). All reagents were purchased from Qiagen (Valencia, Calif.). Single nucleotide polymorphism (SNP) genotype analysis was performed using the PyroMark Q24 software. For statistical analysis multivariate logistic regression analysis was applied to evaluate the differences in SNP distribution, and the odds ratio (OR) and their 95% confidence intervals (CI) was computed using the software JMP9 (SAS).

### Results

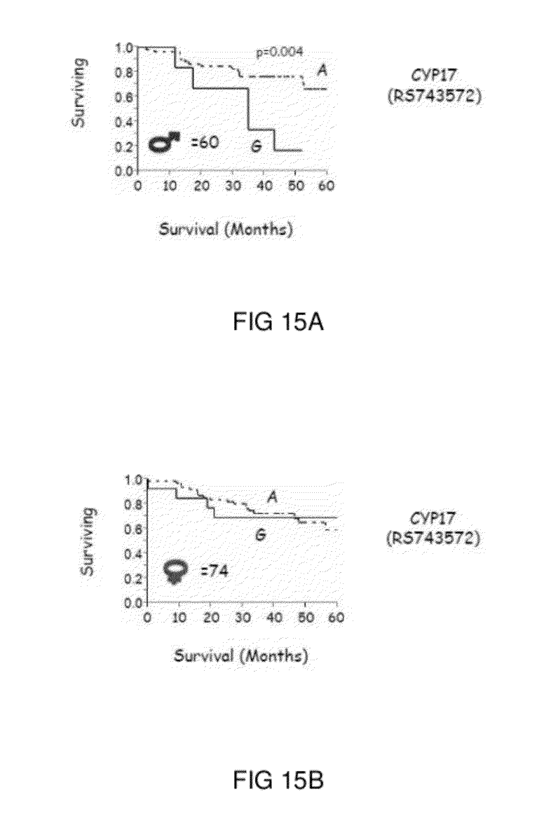

We observed that male patients carrying the G allele exhibited the highest risk of death (OR 10.7, 95% CI, 1.35-86.1), while in females this deleterious effect of the G allele was completely absent (OR 0.98, 95% CI, 0.33-2.87). Kaplan-Meier analysis confirmed the deleterious effect of the G allele only in male patients, showing that this subset progresses rapidly to a fatal outcome, while this fenomenon is not present in females. (FIGS. 15A-B)

1. A method for determining the prognosis of a cancer characterized by a solid tumor in a female patient comprising
in a sample from said solid tumor, determining the percentage of solid tumor cells expressing TUBB3 protein and the percentage of solid tumor cells expressing TUBB6 protein;
wherein detection of TUBB6 protein expression in a percentage of said solid tumor cells that is less than a median percentage expression for TUBB6 protein and detection of TUBB3 protein expression in a percentage of said solid tumor cells that is less than the median percentage expression for TUBB3 protein indicates that the patient has a good prognosis; and
wherein detection of TUBB6 protein expression in a percentage of said solid tumor cells of said female patient that is equal to or greater than a median percentage expression for TUBB6 protein and/or detection of TUBB3 protein expression in a percentage of said solid tumor cells of said female patient that is equal to or greater than the median percentage expression for TUBB3 protein indicates that the patient has a poor prognosis.

2. The method of claim 1 wherein the median percentage expression for TUBB3 protein is determined by determining the percentage of solid tumor cells in a solid tumor sample expressing TUBB3 protein in a plurality of solid tumors, of essentially the same tumor type as the solid tumor of claim 1, taken from patients of both genders who are suffering from said cancer and determining the median;
and wherein the median percentage expression for TUBB6 protein is determined by determining the percentage of solid tumor cells in a solid tumor sample expressing TUBB6 protein in a plurality of solid tumors, of essentially the same tumor type as the solid tumor of claim 1, taken from patients of both genders who are suffering from said cancer and determining the median.

3. The method of claim 1 or 2 wherein said cancer is colorectal cancer or lung cancer.

4. The method of claim 1 or 2 wherein said cancer is colorectal cancer, said median percentage expression for TUBB3 protein is 35% and said median percentage expression for TUBB6 protein is 60%.

5. The method of claim 1 or 2 wherein said solid tumor sample from the patient is a paraffin-embedded sample.

6. The method of claim 1 or 2 wherein said female patient is human.

7. The method of claim 1 or 2 wherein said cancer has a gender-specific prognosis.

8. A method for treating a female patient with a solid tumor comprising administering a chemotherapeutic agent to a female patient following prognosis according to the method of claim 1.

9. A method of sensitizing a cancer cell of a solid tumor of a male patient, other than a prostate cancer cell, to treatment with a chemotherapeutic agent comprising administering to the patient an effective amount of
(a)
(i) at least one antiandrogen;
(ii) at least one inhibitor of androgen synthesis; or
(iii) a combination of at least one antiandrogen and at least one inhibitor of androgen synthesis; and
(b) at least one chemotherapeutic agent other than an antiandrogen or an inhibitor of androgen synthesis.

10. The method of claim 9 comprising administering to the patient an effective amount of at least one antiandrogen and at least one chemotherapeutic agent other than an antiandrogen.

11. The method of claim 9 comprising administering to the patient an effective amount of at least one inhibitor of androgen synthesis and at least one chemotherapeutic agent other than an inhibitor of androgen synthesis.

12. The method of claim 9 wherein said cancer is colorectal cancer or lung cancer.

13. The method of claim 9 wherein said chemotherapy is oxaliplatin or irinotecan.

14. The method of claim 9 wherein said patient is human.

15. The method of claim 9 wherein said cancer cell constitutively expresses TUBB3 protein and TUBB6 protein; and wherein the survival rate of said cancer is worse for males than for females.

16. The method claim 9 wherein said chemotherapeutic agent is administered to said male cancer cell before said antiandrogen or said inhibitor of androgen synthesis.

17. The method of claim 9 wherein said antiandrogen or said inhibitor of androgen synthesis is administered to said cancer cell before said chemotherapeutic agent.

18. The method of claim 9 wherein said chemotherapeutic agent and said antiandrogen or said inhibitor of androgen synthesis are administered to said cancer cell at the same time.

19. The method of claim 9 wherein said antiandrogen is bicalutamide, flutamide, nicalutamide, spironolactone, cyproterone acetate or finasteride.

20. The method of claim 9 wherein said inhibitor of androgen synthesis is an inhibitor of CYP17 or AKR1C3.

21. The method of claim 20 wherein said inhibitor of CYP17 is ketoconazole, abiraterone, abiraterone acetate, VN/124-1(TOK-001) or TAK-700, or salts thereof.

22. The method of claim 20 wherein said inhibitor of AKR1C3 is an indomethacin analog.

23. The method of claim 22 wherein said indomethacin analog is N-(4-chlorobenzoyl)-melatonin.

24. A kit for determining the prognosis of cancer in a female patient with a solid tumor comprising a set of reagents that specifically detects the protein expression levels of TUBB3 protein and TUBB6 protein in cells from said solid tumor, and instructions for using said kit for evaluating the prognosis of cancer characterized by a solid tumor in a female.
